SEQUENCE LISTING (37 sequences)

SEQ ID NO: 1 - protein, 257 aa, Microbispora hainanensis
  source 1..257: note = str. A3051
AAVHGPDPTDALLEASRGPYATRQVDVSSLLVSGFGGGTIYYPTTTSEGTFGGVAIAPGYTADKSSLAWLAARLASHGFVVFNIDTLTRLDQPDSRGRQLLAALDYLTERSSVRGRVDASRLGVMGHSMGGGGTLEAVDGRPSLRAAVPLTPWNLDKTWPGVQTPTLIIGADNDTVAPVSSHAVPFYTSLPSSLDKAYLELNNATHFAPNTTNTTIGKYAVAWMKRFVDDDTRYDRFLCPGPSRGLTVEEYRSTCPF

SEQ ID NO: 2 - protein, 261 aa, Nonomuraea sp.
  source 1..261: note = TT08I-71 str. T589
AADNPYERGPAPTSAILEASRGPFATSSYSVSSLSVTGFGGGVVYYPTSTSEGTFGAIAISPGYTATWSSLSWLGPRIASHGFVVIGIETNTLLDQPDSRGRQLLAALDYLTQRSTVRGRIDASRLAVAGHSMGGGGTLEAAAARPSLQAAVPLAPWNLDKTWNGVTVPTLIVGGESDTIAPVATHSEPFYSSIPASSEKAYLELNGASHFFPQSVNTPMARQMVAWLKRFVDNDTRYEQFLCPGPSGLAIEEYRNTCPSS

SEQ ID NO: 3 - protein, 269 aa, Micromonospora musae
  source 1..269: note = str. T593
AASSGPVQAAENPYERGPAPTVALLEASRGPFSTGSQTVSSLSVTGFGGGVIYYPTDTSQGTFGAIAISPGYTAAWSSISWLGPRIASHGFVVIGIETNSRLDQPASRGRQLLAALDYLVERSSVRTRIDGTRLAVAGHSMGGGGSLEAAASRPSLQAAVPLAPWNLDKTWNTLQVPTLIVGGENDSVAPVASHSIPFYNSIPASAEKAYLELNGASHFFPQTVNTPTARQAVAWLKRFVDDDTRYEQFLCPGPSGVQIEEYRNTCPSA

SEQ ID NO: 4 - protein, 261 aa, Micromonospora chersina
  source 1..261: note = str. A2592
AADNPYERGPAPTSAILEASRGPFATSSYSVSSLSVTGFGGGVVYYPTSTSEGTFGAIAISPGYTATWSSLSWLGPRIASHGFVVIGIETNTLLDQPDSRGRQLLAALDYLTQRSSVRGRIDATRLAVAGHSMGGGGTLEAAAARPSLQAAVPLAPWNLDKTWSGVTVPTLIVGGESDTVAPVATHSEPFYNSIPASSEKAYLELNGASHFFPQSVNTPMAKQMVAWLKRFVDNDTRYEQFLCPGPSGLAIEEYRNTCPSS

SEQ ID NO: 5 - protein, 257 aa, Microbispora camponoti
AAVHGPDPTDALLESSRGPYATAQTDVSSLSASGFGGGTIYYPTTTSEGTFGGVAIAPGYTADKSSLAWLAARLASHGFVVFNIDTLTRLDQPDSRGRQLLAALDYLTQRSSVRGRVDATRLGVMGHSMGGGGTLEAVDDRPSLRAAVPLTPWNLDKTWSGVRTPTLIIGAEADTVAPVVSHAVPFYTSLSSSLDKAYLELNGATHFAPNTTNTTIGKYAVAWMKRFVDDDTRYDQFLCPGPSRSLTVEEYRSTCPF

SEQ ID NO: 6 - protein, 262 aa, Nonomuraea soli
ASASPYQRGPDPTEAVLEASRGPFSVSDTTVSSLVVTGFGGGTIYYPTSTAEGTFGGVAIAPGYTASRSSMAWLAERIASHGFVVFNIDTITRSDQPASRGRQLLAALDYLTERSTVRSRLDASRLGVMGHSMGGGGTLEASASRPSLQAAVPLTPWNLDKSWSEVQVPTMIIGAENDSIASVTSHSEPFYNSLPSSLDKAYLELDGASHFAPNTTNPTIGKFSVAWLKRFIDDDLRYEQFLCPPPVGDRDISEYRNTCPHS

SEQ ID NO: 7 - protein, 260 aa, Microbispora sp.
AAHAAVHGPDPTDALLEASRGPYATAQVDVSSLLVSGFGGGTIYYPATTSEGTFGGVAIAPGYTADKSSLAWLAARLASHGFVVFNIDTLTRLDQPDSRGRQLLAALDYLTQRSSVRGRVDATRLGVMGHSMGGGGTLEAVDDRPSLRAAVPLTPWNLDKTWSGVRTPTLIIGADNDTIAPVASHAVPFYTSLPSSLDKAYLELNNATHFAPNTTNTTIGKYAVAWMKRFIDDDTRYDGFLCPGPDRGLTVEEYRSTCPF

SEQ ID NO: 8 - protein, 267 aa, synthetic construct
MSNPYQRGPNPTRSALTADGPFSVATYTVSRLSVSGFGGGVIYYPTGTSLTFGGIAMSPGYTADASSLAWLGRRLASHGFVVLVINTNSRFDGPDSRASQLSAALNYLRTSSPSAVRARLDANRLAVAGHAMGGGGTLRIAEQNPSLKAAVPLTPWHTDKTFNTSVPVLIVGAEADTVAPVSQHAIPFYQNLPSTTPKVYVELCNASHIAPNSNNAAISVYTISWMKLWVDNDTRYRQFLCNVNDPALCDFRTNNRHCQLEHHHHHH

SEQ ID NO: 9 - protein, 286 aa, synthetic construct
MGSSHHHHHHAGAAAVHGPDPTDALLEASRGPYATRQVDVSSLLVSGFGGGTIYYPTTTSEGTFGGVAIAPGYTADKSSLAWLAARLASHGFVVFNIDTLTRLDQPDSRGRQLLAALDYLTERSSVRGRVDASRLGVMGHSMGGGGTLEAVDGRPSLRAAVPLTPWNLDKTWPGVQTPTLIIGADNDTVAPVSSHAVPFYTSLPSSLDKAYLELNNATHFAPNTTNTTIGKYAVAWMKRFVDDDTRYDRFLCPGPSRGLTVEEYRSTCPFAGAHHHHHHSEQIDNO

SEQ ID NO: 10 - protein, 283 aa, synthetic construct
MGSSHHHHHHAGAAADNPYERGPAPTSAILEASRGPFATSSYSVSSLSVTGFGGGVVYYPTSTSEGTFGAIAISPGYTATWSSLSWLGPRIASHGFVVIGIETNTLLDQPDSRGRQLLAALDYLTQRSTVRGRIDASRLAVAGHSMGGGGTLEAAAARPSLQAAVPLAPWNLDKTWNGVTVPTLIVGGESDTIAPVATHSEPFYSSIPASSEKAYLELNGASHFFPQSVNTPMARQMVAWLKRFVDNDTRYEQFLCPGPSGLAIEEYRNTCPSSAGAHHHHHH

SEQ ID NO: 11 - protein, 293 aa, synthetic construct
MVSEPHDYNYEKAGAAASSGPVQAAENPYERGPAPTVALLEASRGPFSTGSQTVSSLSVTGFGGGVIYYPTDTSQGTFGAIAISPGYTAAWSSISWLGPRIASHGFVVIGIETNSRLDQPASRGRQLLAALDYLVERSSVRTRIDGTRLAVAGHSMGGGGSLEAAASRPSLQAAVPLAPWNLDKTWNTLQVPTLIVGGENDSVAPVASHSIPFYNSIPASAEKAYLELNGASHFFPQTVNTPTARQAVAWLKRFVDDDTRYEQFLCPGPSGVQIEEYRNTCPSAAGAHHHHHH

SEQ ID NO: 12 - protein, 408 aa, synthetic construct
MGGGSSEHEHEHSSGLVPRGSHMASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSAGAETASSEGLAGAAADNPYERGPAPTSAILEASRGPFATSSYSVSSLSVTGFGGGVVYYPTSTSEGTFGAIAISPGYTATWSSLSWLGPRIASHGFVVIGIETNTLLDQPDSRGRQLLAALDYLTQRSSVRGRIDATRLAVAGHSMGGGGTLEAAAARPSLQAAVPLAPWNLDKTWSGVTVPTLIVGGESDTVAPVATHSEPFYNSIPASSEKAYLELNGASHFFPQSVNTPMAKQMVAWLKRFVDNDTRYEQFLCPGPSGLAIEEYRNTCPSSAGAHHHHHH

SEQ ID NO: 13 - protein, 279 aa, synthetic construct
MGSSHHHHHHAGAAAVHGPDPTDALLEASRGPYATRQVDVSSLLVSGFGGGTIYYPTTTSEGTFGGVAIAPGYTADKSSLAWLAARLASHGFVVFNIDTLTRLDQPDSRGRQLLAALDYLTERSSVRGRVDASRLGVMGHSMGGGGTLEAVDGRPSLRAAVPLTPWNLDKTWPGVQTPTLIIGADNDTVAPVSSHAVPFYTSLPSSLDKAYLELCNATHIAPNTTNTTIGKYAVAWMKRFVDDDTRYDRFLCPGPSRGLCVEEYRSTCPFAGAHHHHHH

SEQ ID NO: 14 - protein, 279 aa, synthetic construct
MGSSHHHHHHAGAAAVHGPDPTDALLEASRGPYATRQVDVSSLLVSGFGGGTIYYPTTTSEGTFGGVAIAPGYTADKSSLAWLAARLASHGFVVFNIDTLTRLDQPDSRGRQLLAALDYLTERSSVRGRVDASRLGVMGHSMGGGGTLEAVDGRPSLRAAVPLTPWNLDKTWPGVQTPTLIIGADNDTVAPVSSHAVPFYTSLPSSLDKAYLELCNATHIAPMTTNTTIGKYAVAWMKRFVDDDTRYDRFLCPGPSRGLCVEEYRSTCPFAGAHHHHHH

SEQ ID NO: 15 - protein, 682 aa, synthetic construct
MKYYKHEHEHEHEHEHEHEGASGMKIEEGKVVIWHAMQPNELEVFQSLAEEYMALCPEVEIVFEQKPNLEDALKAAIPTGQGPDLFIWAHDWIGKFAEAGLLEPIDEYVTEDLLNEFAPMAQDAMQYKGHYYALPFAAETVAIIYNKEMVSEPPKTFDEMKAIMEKYYDPANEKYGIAWPINAYFISAIAQAFGGYYFDDKTEQPGLDKPETIEGFKFFFTEIWPYMAPTGDYNTQQSIFLEGRAPMMVNGPWSINDVKKAGINFGVVPLPPIIKDGKEYWPRPYGGVKLIYFAAGIKNKDAAWKFAKWLTTSEESIKTLALELGYIPVLTKVLDDPEIKNDPVIYGFGQAVQHAYLMPKSPKMSAVWGGVDGAINEILQDPQNADIEGILKKYQQEILNNMQGEENLYFQSIAGAAAVHGPDPTDALLESSRGPYATAQTDVSSLSASGFGGGTIYYPTTTSEGTFGGVAIAPGYTADKSSLAWLAARLASHGFVVFNIDTLTRLDQPDSRGRQLLAALDYLTQRSSVRGRVDATRLGVMGHSMGGGGTLEAVDDRPSLRAAVPLTPWNLDKTWSGVRTPTLIIGAEADTVAPVVSHAVPFYTSLSSSLDKAYLELCGATHIAPMTTNTTIGKYAVAWMKRFVDDDTRYDQFLCPGPSRSLCVEEYRSTCPFAGAHHHHHH

SEQ ID NO: 16 - protein, 687 aa, synthetic construct
MKYYKHEHEHEHEHEHEHEGASGMKIEEGKVVIWHAMQPNELEVFQSLAEEYMALCPEVEIVFEQKPNLEDALKAAIPTGQGPDLFIWAHDWIGKFAEAGLLEPIDEYVTEDLLNEFAPMAQDAMQYKGHYYALPFAAETVAIIYNKEMVSEPPKTFDEMKAIMEKYYDPANEKYGIAWPINAYFISAIAQAFGGYYFDDKTEQPGLDKPETIEGFKFFFTEIWPYMAPTGDYNTQQSIFLEGRAPMMVNGPWSINDVKKAGINFGVVPLPPIIKDGKEYWPRPYGGVKLIYFAAGIKNKDAAWKFAKWLTTSEESIKTLALELGYIPVLTKVLDDPEIKNDPVIYGFGQAVQHAYLMPKSPKMSAVWGGVDGAINEILQDPQNADIEGILKKYQQEILNNMQGEENLYFQSIAGAASASPYQRGPDPTEAVLEASRGPYSVSDTTVSSLLVSGFGGGTIYYPTSTAEGTFGGVAIAPGYTASRSTMAWLAERITSHGFVVFNIDTITRSDQPASRGRQLLAALDYLTERSSVRSRLDASRLGVMGHSMGGGGTLEASSSRPSLQAAVPLTPWNLDKSWSEVQVPTMIIGAENDSIASVTTHSEPFYNSLPSSLDKAYLELCGASHIAPMTTNPTIGKFSVAWLKRFIDDDLRYEQFLCPPPVGGLCISEYRNTCPHSAGAHHHHHH

SEQ ID NO: 17 - protein, 257 aa, synthetic construct
AAVHGPDPTDALLEASRGPYATRQVDVSSLLVSGFGGGTIYYPTTTSEGTFGGVAIAPGYTADKSSLAWLAARLASHGFVVFNIDTLTRLDQPDSRGRQLLAALDYLTERSSVRGRVDASRLGVMGHSMGGGGTLEAVDGRPSLRAAVPLTPWNLDKTWPGVQTPTLIIGADNDTVAPVSSHAVPFYTSLPSSLDKAYLELCNATHIAPMTTNTTIGKYAVAWMKRFVDDDTRYDRFLCPGPSRGLCVEEYRSTCPF

SEQ ID NO: 18 - protein, 257 aa, synthetic construct
AAVHGPDPTDALLESSRGPYATAQTDVSSLSASGFGGGTIYYPTTTSEGTFGGVAIAPGYTADKSSLAWLAARLASHGFVVFNIDTLTRLDQPDSRGRQLLAALDYLTQRSSVRGRVDATRLGVMGHSMGGGGTLEAVDDRPSLRAAVPLTPWNLDKTWSGVRTPTLIIGAEADTVAPVVSHAVPFYTSLSSSLDKAYLELCGATHIAPMTTNTTIGKYAVAWMKRFVDDDTRYDQFLCPGPSRSLCVEEYRSTCPF

SEQ ID NO: 19 - protein, 262 aa, synthetic construct
ASASPYQRGPDPTEAVLEASRGPYSVSDTTVSSLLVSGFGGGTIYYPTSTAEGTFGGVAIAPGYTASRSTMAWLAERITSHGFVVFNIDTITRSDQPASRGRQLLAALDYLTERSSVRSRLDASRLGVMGHSMGGGGTLEASSSRPSLQAAVPLTPWNLDKSWSEVQVPTMIIGAENDSIASVTTHSEPFYNSLPSSLDKAYLELCGASHIAPMTTNPTIGKFSVAWLKRFIDDDLRYEQFLCPPPVGGLCISEYRNTCPHS

SEQ ID NO: 20 - protein, 257 aa, synthetic construct
AAVHGPDPTDALLEASRGPYATRQVDVSSLLVSGFGGGTIYYPTTTSEGTFGGVAIAPGYTADASSLAWLAARLASHGFVVFNIDTLTRFDYPDSRGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTTNTTIGKYAVAWMKRFVDDDTRYDRFLCPGPSRGLCVEEYRSTCPF

SEQ ID NO: 21 - protein, 257 aa, synthetic construct
AAVKGPDPTDALLEASRGPYATRQVDVSSLLVSGFGGGTIYYPTTTSEGTFGGVAIAPGYTADASSLAWLAARLASHGFVVFNIDTLTRFDYPDSRGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTTNTTIGKYAVAWMKRFVDDDTRYDRFLCPGPSRGLCVEEYRSTCPF

SEQ ID NO: 22 - protein, 257 aa, synthetic construct
AAVHGPDPTDALLEASRGPYATRQVDVSSALVSGFGGGTIYYPTTTSEGTFGGVAIAPGYTADASSLAWLAARLASHGFVVFNIDTLTRFDYPDSRGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTTNTTIGKYAVAWMKRFVDDDTRYDRFLCPGPSRGLCVEEYRSTCPF

SEQ ID NO: 23 - protein, 257 aa, synthetic construct
AAVHGPDPTDALLEASRGPYATRQVDVSSLAVSGFGGGTIYYPTTTSEGTFGGVAIAPGYTADASSLAWLAARLASHGFVVFNIDTLTRFDYPDSRGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTTNTTIGKYAVAWMKRFVDDDTRYDRFLCPGPSRGLCVEEYRSTCPF

SEQ ID NO: 24 - protein, 257 aa, synthetic construct
AAVHGPDPTDALLEASRGPYATRQVDVSSLLVSGFGGGTIYYPTTTSEGTFGGVAIAPGYGADASSLAWLAARLASHGFVVFNIDTLTRFDYPDSRGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTTNTTIGKYAVAWMKRFVDDDTRYDRFLCPGPSRGLCVEEYRSTCPF

SEQ ID NO: 25 - protein, 257 aa, synthetic construct
AAVHGPDPTDALLEASRGPYATRQVDVSSLLVSGFGGGTIYYPTTTSEGTFGGVAIAPGYTADASSLAWLAARLASHGFVVFNIDTLTRFDGPDSRGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTTNTTIGKYAVAWMKRFVDDDTRYDRFLCPGPSRGLCVEEYRSTCPF

SEQ ID NO: 26 - protein, 257 aa, synthetic construct
AAVHGPDPTDALLEASRGPYATRQVDVSSLLVSGFGGGTIYYPTTTSEGTFGGVAIAPGYTADASSLAWLAARLASHGFVVFNIDTLTRFDYPDARGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTTNTTIGKYAVAWMKRFVDDDTRYDRFLCPGPSRGLCVEEYRSTCPF

SEQ ID NO: 27 - protein, 257 aa, synthetic construct
AAVHGPDPTDALLEASRGPYATRQVDVSSLLVSGFGGGTIYYPTTTSEGTFGGVAIAPGYTADASSLAWLAARLASHGFVVFNIDTLTRFDYPDSRGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTANTTIGKYAVAWMKRFVDDDTRYDRFLCPGPSRGLCVEEYRSTCPF

SEQ ID NO: 28 - protein, 257 aa, synthetic construct
AAVHGPDPTDALLEASRGPYATRQVDVSSLLVSGFGGGTIYYPTTTSEGTFGGVAIAPGYTADASSLAWLAARLASHGFVVFNIDTLTRFDYPDSRGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTTNTTIAKYAVAWMKRFVDDDTRYDRFLCPGPSRGLCVEEYRSTCPF

SEQ ID NO: 29 - protein, 257 aa, synthetic construct
AAVHGPDPTDALLEASRGPYATRQVDVSSLLVSGFGGGTIYYPTTTSEGTFGGVAIAPGYTADASSLAWLAARLASHGFVVFNIDTLTRFDYPDSRGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTTNTTIGKYVVAWMKRFVDDDTRYDRFLCPGPSRGLCVEEYRSTCPF

SEQ ID NO: 30 - protein, 257 aa, synthetic construct
AAVHGPDPTDALLEASRGPYATRQVDVSSLLVSGFGGGTIYYPTTTSEGTFGGVAIAPGYTADASSLAWLAARLASHGFVVFNIDTLTRFDYPDSRGRQLLAALDYLTERSSVRGRVDASRLGVMGHSMGGGGTLRAVDGRPSLRAAVPLTPWHLDKTWPGVQTPTLIIGADNDTVAPVSSHAVPFYTSLPSSLDKAYLELCNATHIAPMTTNTTIGKYAVAWMKRFVDDDTRYDRFLCPGPSRGACVEEYRSTCPF

SEQ ID NO: 31 - protein, 13 aa, synthetic construct
MGSSHHHHHHAGA

SEQ ID NO: 32 - protein, 138 aa, synthetic construct
MGGGSSEHEHEHSSGLVPRGSHMASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSAGAETASSEGLAGA

SEQ ID NO: 33 - protein, 15 aa, synthetic construct
MVSEPHDYNYEKAGA

SEQ ID NO: 34 - protein, 415 aa, synthetic construct
MQLSHEHEHEHEHEHEHEGASGMKIEEGKVVIWHAMQPNELEVFQSLAEEYMALCPEVEIVFEQKPNLEDALKAAIPTGQGPDLFIWAHDWIGKFAEAGLLEPIDEYVTEDLLNEFAPMAQDAMQYKGHYYALPFAAETVAIIYNKEMVSEPPKTFDEMKAIMEKYYDPANEKYGIAWPINAYFISAIAQAFGGYYFDDKTEQPGLDKPETIEGFKFFFTEIWPYMAPTGDYNTQQSIFLEGRAPMMVNGPWSINDVKKAGINFGVVPLPPIIKDGKEYWPRPYGGVKLIYFAAGIKNKDAAWKFAKWLTTSEESIKTLALELGYIPVLTKVLDDPEIKNDPVIYGFGQAVQHAYLMPKSPKMSAVWGGVDGAINEILQDPQNADIEGILKKYQQEILNNMQGEENLYFQSIAGA

SEQ ID NO: 35 - protein, 93 aa, synthetic construct
AGAEKEKEKEKEKEKEKEKEKEKEKEKEKEKEKEKEKEKEKEKEKEKEKEKEKEKEKEKEKEKEKEKEKEKEKEKEKEKEKEKEKEKEKEKEK

SEQ ID NO: 36 - protein, 13 aa, synthetic construct
MYPYDVPDYAAGA

SEQ ID NO: 37 - protein, 19 aa, synthetic construct
DVFKKGFSIADEGVVGAVE

(19) United States
(12) Patent Application Publication
(10) Pub. No.: US 2026/0357229 A1
(43) Pub. Date: Nov. 12, 2026

(54) Microbial polypeptides and uses thereof

(71) Applicant: Agency for Science, Technology and Research, Singapore (SG)

(72) Inventors: Fong Tian Wong, Singapore (SG); Elaine Pei Ying Tiong, Singapore (SG); Lokanand Koduru, Singapore (SG); Yee Hwee Lim, Singapore (SG); Ying Sin Koo, Singapore (SG)

(21) Appl. No.: 19/471,353

(22) PCT Filed: Apr. 5, 2024
(86) PCT No.: PCT/SG2024/050234

§ 371 Date: Sep. 30, 2025

(30) Foreign Application Priority Data
Apr. 6, 2023 (SG) 10202300968P

(51) Int. Cl. C12N 9/18

(52) U.S. Cl. CPC C12N 9/18

(57) ABSTRACT

The present invention relates to polypeptides for degrading polyesters. Disclosed herein are microbially-derived polypeptides and engineered variants thereof for degrading polyethylene terephthalate (PET) and other polyesters. Also provided are methods of producing the polypeptides and methods for polyester degradation using the polypeptides.

### INCORPORATION BY REFERENCE

The XML file “DAV10SeqListing.XML” created on Jul. 20, 2026, having the size of 43,187 bytes, is incorporated by reference into the specification.

### TECHNICAL FIELD

The present invention relates to polypeptides for degrading polyesters, and uses thereof.

### BACKGROUND

Polyethylene terephthalate (PET) is a synthetic polyester and is among the most widely manufactured and utilised plastic material for consumer and industrial applications due to its durability and favourable physicochemical properties. However, the rapid accumulation of post-consumer PET waste, which is largely non-biodegradable, poses risks to human health and to the environment. Traditional solid waste treatment methods, such as landfill and incineration, are pollutive. While PET can be recycled, conventional thermomechanical and chemical recycling methods are energy-intensive, and can compromise the properties of the plastic material, making it unsuitable for re-processing. There is thus a need for more environmentally sustainable methods of processing and recycling PET and other polyester-based plastics.

It would be desirable to overcome or alleviate at least one of the above-described problems, or at least to provide a useful alternative.

### SUMMARY

Disclosed herein is an isolated polypeptide that is distinguished from a wild-type hydrolase enzyme by at least one amino acid substitution, wherein the at least one amino acid substitution is at a position corresponding to position 202, 247, 207, 210, 64, 90, 92, 154, 61 or 212 of SEQ ID NO: 1.

Disclosed herein is an isolated polypeptide comprising an amino acid sequence having at least 70% sequence identity to an amino acid sequence set forth in SEQ ID NO: 1, SEQ ID NO: 2, SEQ ID NO: 3 or SEQ ID NO: 4, SEQ ID NO: 5, SEQ ID NO: 6 or SEQ ID NO: 7.

Disclosed herein is an isolated polypeptide comprising an amino acid sequence that is distinguished from an amino acid sequence of SEQ ID NO: 1-7 by at least one amino acid substitution, wherein the at least one amino acid substitution is at a position corresponding to position 4, 30, 31, 61, 64, 90, 92, 95, 136, 154, 202, 207, 210, 212, 217, 220, 246 or 247 of SEQ ID NO: 1.

Disclosed herein is a polynucleotide encoding a polypeptide as defined herein.

Disclosed herein is an expression vector comprising a polynucleotide as defined herein.

Disclosed herein is a host cell comprising a polynucleotide or expression vector as defined herein.

Disclosed herein is a method of producing a polypeptide as defined herein, comprising culturing a host cell as defined herein under conditions suitable for expressing the polypeptide.

Disclosed herein is a method of degrading a product comprising at least one polyester, the method comprising contacting the product with a polypeptide as defined herein and/or with a cell expressing a polypeptide as defined herein under conditions suitable for degrading the at least one polyester.

Disclosed herein is a kit, comprising a polypeptide as defined herein.

### BRIEF DESCRIPTION OF THE DRAWINGS

Embodiments of the present invention will now be described, by way of non-limiting example, with reference to the drawings in which:

FIG. 1 shows genomic mining and in silico analysis of distant cutinase homologs. (A) Structural alignment of LCC and IsPETase against AlphaFold models of SIBER 1-4. Active sites residues are shown in inset below. RSMD of the in-silico models against 6THS range from 0.54 to 0.68 Å. (B) Similarity scores between SIBER 1-4, LCC and IsPETase using BLASTP. (C) Electrostatic representative of solvent accessible surfaces of LCC and SIBER 1 at pH7 (±7 kT/e, APBS-PDB2PQR).

FIG. 2 shows protein expression optimization for SIBER 1-4. Heat map of expression levels of SIBER 1-4 constructs with various solubility tags in E. coli. The proteins are his-tagged purified and subsequently separated using a SDS-PAGE protein gel. (A) Heat map of SIBER 1-4 constructs. The expression level of proteins is determined by the degree of darkness, where white boxes represent proteins that have the highest expression while black represents proteins that have low or no expression. Quantification was made using ImageJ and normalized against the ladder. Expression of SIBER 171, SIBER 196, SIBER 207, SIBER 228 are annotated as (a)-(d) respectively. (B)-(C) Expression bar graphs of enzymes, where SIBER 1102 and SIBER 1118 were selected for further characterisation. Amino acid sequence of the protein of interest can be found in Table 3.

FIG. 3 shows characterisation of SIBER 1-4. Depolymerisation of 5 mM BHET for (A) 24 hours at various temperatures, pH 7, or (B) 24 hours at various pH, 25 °C. Y axis (%) and X axis (mutant numbering SIBER-). (C) Depolymerisation of high crystallinity PET (Goodfellow biaxially oriented film) at 25 °C and 45 °C over 2 weeks. (D) Depolymerisation of low crystallinity PET at 45 °C, pH 7 over 72 hours.

FIG. 4 shows characterisation of engineered SIBER 1. (A) Depolymerisation of 5 mM BHET for 50 °C, pH 7, 24 hours. (B) Depolymerisation of low crystallinity PET (Goodfellow amorphous film) at 45 °C over 48 hours and 1 week. Y axis (%) and X axis (mutant numbering SIBER-).

FIG. 5 shows sequence alignment of optimized LCC against SIBER 1-4, structural elements based on PDB 6THS are annotated. Clustal Omega and ESPript 3 were used to generate the alignment.

FIG. 6 shows sequence similarity networks of SIBER 1. (A) SIBER 1 amino acid sequence was put through BLAST against UniProt protein database using the EFI-EST online tool to generate a sequence similarity network (SSN). The various clusters of bacterial proteins belong to three major bacterial phyla: proteobacteria, firmicutes and actinobacteria which SIBER proteins, LCC and PETase are clustered together with. Within the major actinobacteria cluster, SIBER 1 can be observed to be far away from LCC and PETase. Compared to SIBER 2-4, SIBER 1 is shown to be more similar to SIBER 2 and 4, with a similarity comparable to that of PETase in relation to SIBER 3. Despite being in the same supercluster, there are significant differences between the engineered SIBER proteins and that of wild type enzymes given the scale of the SSN, which included proteins from eukaryotic sources. (B) Details of sequence similarity networks of SIBER 1 with related enzymes.

FIG. 7 is an SDS-PAGE analysis of purified SIBER 171. Proteins were separated using a 4-20% Mini-Protean TGX Stain-Free Precast gel (Bio-rad), and the bands obtained were referenced to the Novex Sharp Unstained Protein Standard (Invitrogen).

FIG. 8 shows the depolymerisation yield of low crystallinity PET (lcPET) with cell lysate containing SIBER 1136 and SIBER822 at 45 °C over 48 h. The table in (B) shows the proportion of PET degradation products.

FIG. 9 shows the depolymerisation yield of lcPET with purified SIBER 1136 and LCC at 45 °C over 2 weeks.

FIG. 10 shows the fold-change in depolymerisation yield of lcPET using various SIBER mutants compared to SIBER 1136 (fold change=1) at pH 8, 45 °C, 24 h. This experiment was conducted in a high throughput 1 mL screening format.

FIG. 11 shows the expression yield of SIBER1 variants (SIBER 1136 and SIBER 822) compared to LCC-ICCG (average is given by the horizontal line and individual data points are shown as filled circles).

FIG. 12 shows depolymerisation of low crystallinity PET using SIBER 1102 (Nonomuraea soli, top panel) and SIBER 1118 (Microbispora camponoti, bottom panel) at 70 °C for 6 hours. The LC spectra extracted indicate peaks corresponding to TPA (RT 8 min and 8.45 min). A control reaction carried out in the absence of enzymes was used for comparison.

FIG. 13 shows depolymerisation of BHET using purified SIBER 1 variants with different solubility tags. SIBER 748 contains MBP tagged SIBER 1 and SIBER 755 contains HA tagged SIBER 1.

FIG. 14 shows depolymerisation of low crystallinity PET with SIBER 1 variants with different solubility tags. Eluate from 1 mL high throughput expression were used for degradation studies. SIBER 1128 contains NT11 tagged SIBER 1 and SIBER 1136 contains His tagged SIBER 1.

FIG. 15 shows depolymerisation of low crystallinity PET with two SIBER 1 variants containing VNp15 tag.

### DETAILED DESCRIPTION

The inventors have isolated and characterised several new polyester-degrading enzymes from the Microbispora, Nonomuraea and Micromonospora genera through mining of actinobacterial genomic sequences. Advantageously, enzyme-catalysed processing of PET and other polyesters can proceed under mild conditions and is thus more environmentally sustainable than conventional thermomechanical and chemical polyester recycling methods. The inventors have further engineered the enzymes for improved thermostability, catalytic efficacy and recombinant expression through site-directed mutagenesis.

Accordingly, this specification discloses isolated wild-type and engineered microbial polypeptides which are capable of degrading polyesters (e.g., polyethylene terephthalate) under mild conditions, methods of producing the polypeptides, and methods of using the polypeptides for polyester degradation.

### Polyester-Degrading Enzymes

Disclosed herein is an isolated polypeptide comprising an amino acid sequence having at least 70% sequence identity to an amino acid sequence set forth in SEQ ID NO: 1, SEQ ID NO: 2, SEQ ID NO: 3 or SEQ ID NO: 4, SEQ ID NO: 5, SEQ ID NO: 6, or SEQ ID NO: 7.

The polypeptide may comprise an amino acid sequence having at least 70% sequence identity, at least 75%, at least 80%, at least 85%, at least 90%, at least 91%, at least 92%, at least 93%, at least 94%, at least 95%, at least 96%, at least 97%, at least 98% or at least 99% sequence identity to an amino acid sequence set forth in SEQ ID NO: 1-7. Sequence variations herein include amino acid substitutions, insertions, deletions and sequence inversions, and may be natural or engineered. It is to be understood that the sequence variations herein (such as the up to 1%, 2%, 3%, 4%, 5%, 6%, 7%, 8%, 9%, 10%, 15%, 20%, 25% or 30% sequence variation) are variations which do not substantially affect any biological activity of the polypeptide. For example, the variations may be conservative amino acid substitutions and/or are located in segments of the polypeptide which do not contain the following: an active site, an allosteric site, a chelating site, a site for protein modification (e.g., a phosphorylation, acetylation, glycosylation or cleavage site), a site for intramolecular interaction (e.g., a site of a disulphide or other covalent or non-covalent bond), a binding site for a receptor, ligand, antigen, nucleic acid, protein, lipid, ion or metabolite, a site for an intermolecular covalent or non-covalent interaction, or a multimerisation site (including a dimerisation site). A skilled person can identify appropriate segments and sites which minimally affect a biological activity of a polypeptide using, for example, structural or homology data for the polypeptide.

The polypeptide may comprise 1-10 amino acid deletions at the N- or C-terminus.

In some embodiments, the polypeptide comprises an amino acid sequence set forth in SEQ ID NO: 1-7.

The terms “polypeptide”, “proteinaceous molecule”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogs of an amino acid including, for example, unnatural amino acids or polypeptides with substituted linkages.

As used herein “sequence identity” refers to the number (or fraction expressed as a percentage %) of identical or similar amino acids or nucleotide bases in a comparison between a test and a reference polypeptide or polynucleotide. Sequence identity can be determined by sequence alignment of nucleic acid or protein sequences to identify regions of similarity or identity. For purposes herein, sequence identity is generally determined by alignment to identify identical residues. Alignment can be local or global, but for purposes herein alignment is generally a global alignment where the full-length of each sequence is compared. Matches, mismatches and gaps can be identified between compared sequences. Gaps are null amino acids or nucleotides inserted between the residues of aligned sequences so that identical or similar characters are aligned. Generally, there can be internal and terminal gaps. Sequence identity can be determined by taking into account gaps as the number of identical residues/length of the shortest sequence×100. When using gap penalties, sequence identity can be determined with no penalty for end gaps (e.g., terminal gaps are not penalised). Alternatively, sequence identity can be determined without taking into account gaps as the number of identical positions/length of the total aligned sequence×100.

Alignment for purposes of determining percent amino acid sequence identity can be achieved in various ways that are known to those skilled in the art, for instance, using publicly available computer software available on internet web sites such as http://blast.ncbi.nlm.nih.gov/ or http://www.ebi.ac.uk/Tools/emboss/). Those skilled in the art can determine appropriate parameters for measuring alignment, including any algorithms needed to achieve maximal alignment over the full length of the sequences being compared.

As used herein, the phrase “consisting essentially of” in the context of a recited subunit sequence (e.g., amino acid sequence) indicates that the sequence may comprise at least one additional upstream subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more upstream subunits; e.g., amino acids) and/or at least one additional downstream subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more upstream subunits; e.g., amino acids), wherein the number of upstream subunits and the number of downstream subunits are independently selectable.

In some embodiments, the polypeptide comprises a fusion partner. The fusion partner may be a peptide or polypeptide. The fusion partner may be at the N- or C-terminus of the polypeptide. In preferred embodiments, the fusion partner is recombinantly attached to the polypeptide and is expressed together with the polypeptide. The fusion partner may be added for any purpose, including but not limited to facilitating purification, improving manufacturability, enhancing protein biophysical properties (e.g., solubility or stability), enabling protein labelling for identification, etc. For instance, a hexahistidine tag may be added to enable affinity chromatography during protein purification, or a solubility tag may be added to enhance protein solubility during expression.

In some embodiments the fusion partner is capable of improving the solubility of the polypeptide, e.g., during polypeptide expression or purification. Exemplary fusion partners that can improve solubility include, but are not limited to, peptides comprising histidine repeats (e.g., hexahistidine sequences); NT11 peptide (VSEPHDYNYEK); polyionic peptides comprising repeats of glutamic acid (E) and lysine (K); thioredoxin protein or a fragment thereof, maltose binding protein (MBP); SUMO protein or a fragment thereof, and vesicle nucleating peptides (Eastwood et al, Cell Rep Methods, 2023, 3(2): 100396).

In some embodiments the fusion partner comprises an amino acid sequence set forth in SEQ ID NO: 31-37.

In some embodiments the polypeptide comprises an amino acid sequence having at least 70% sequence identity to an amino acid sequence set forth in SEQ ID NO: 9-12. In some embodiments the polypeptide comprises an amino acid sequence having at least 70%, at least 75%, at least 80%, at least 85%, at least 90%, at least 91%, at least 92%, at least 93%, at least 94%, at least 95%, at least 96%, at least 97%, at least 98% or at least 99% sequence identity to an amino acid sequence set forth in SEQ ID NO: 9-12.

In some embodiments, the polypeptide has hydrolase activity. In some embodiments the polypeptide is capable of hydrolysing ester bonds. In some embodiments the polypeptide is a carboxylic-ester hydrolase.

As used herein a hydrolase is an enzyme in the class of EC 3 hydrolases defined by the Nomenclature Committee of the International Union of Biochemistry and Molecular Biology (NC-IUBMB), and listed in the NC-IUBMB enzyme database, which is accessible at the following weblink: https://www.enzyme-database.org/class.php. Hydrolases include esterases (EC 3.1), glycosylases (EC 3.2), peptidases (EC 3.4), etc. A carboxylic-ester hydrolase is an enzyme in the class of EC 3.1.1 hydrolases defined by the NC-IUBMB.

The polypeptide may be capable of degrading natural or synthetic esters of a range of molecular weights, e.g., monoesters, diesters, oligomeric esters or polymeric esters. As used herein a “monoester” refers to a compound with a single ester bond, and a “diester” refers to a compound with two ester bonds. An “polymeric ester” or “polyester” herein refers to a compound or mixture of compounds whose structure is constituted of multiple monomers (repeat units) linked by ester bonds. A polyester may be constituted of a single type of repeat unit (i.e., a homopolymer) or of a mixture of different repeat units (i.e., a copolymer or heteropolymer). Heteropolymeric polyesters may be produced from the polycondensation of a dicarboxylic acid and a diol. An “oligomeric ester” herein refers to a molecule containing from 2 to about 20 monomers linked by ester bonds.

“Degradation” or a “degrading process” herein refers to a process in which a polymer is broken down to smaller molecules, such as oligomers, monomers, oligomeric or monomeric derivatives, water or carbon dioxide.

In some embodiments the polypeptide is capable of degrading aliphatic esters, aromatic esters, semi-aromatic esters or a combination thereof. An “aromatic ester” is an ester compound derived solely from aromatic monomers. An “aliphatic ester” is a ester compound derived solely from non-aromatic monomers. A “semi-aromatic ester” is an ester comprising at least one aromatic monomer.

In some embodiments the polypeptide is capable of degrading a polyester. Polyesters may be produced by polycondensation of a dicarboxylic acid component and a diol component. For example, the semi-aromatic polyester polyethylene terephthalate (PET) may be produced by polycondensation of terephthalic acid (TPA) as the dicarboxylic acid component and ethylene glycol (EG) as the diol component. Other aromatic dicarboxylic acid components include but are not limited to phthalic acid, isophthalic acid, diphenyldicarboxylic acid, diphenoxyethanedicarboxylic acid, 2,5-naphthalenedicarboxylic acid and derivatives thereof. Examples of aliphatic dicarboxylic acids used in the production of aliphatic polyesters include but are not limited to succinic acid, adipic acid, azelaic acid, sebacic acid, decanedicarboxylic acid and derivatives thereof. Examples of diol components other than ethylene glycol which may also be used to produce polyesters include diethylene glycol, trimethylene glycol, tetramethylene glycol, propylene glycol, pentamethylene glycol, hexamethylene glycol, decamethylene glycol and the like.

Polyester degradation may produce a mixture of oligomers, monomers and their derivatives. In particular, enzymatic degradation of PET may produce a mixture comprising the diesters bis(2-hydroxyethyl terephthalate) (BHET) and dimethyl terephthalate (DMT), the monoesters mono(2-hydroxyethyl) terephthalate (MHET) and 2-hydroxyethyl benzoate (HEB), and the constitutent monomers terephthalic acid (TPA) and ethylene glycol (EG). Cutinases like Thermobifida fusca cutinase usually give TPA and EG as the main breakdown products. PETases like Ideonella sakaiensis PETase may give MHET as the main breakdown product, with trace amounts of BHET, TPA and EG. MHETases like Ideonella sakaiensis MHETase further degrade MHET to TPA and EG. The enzymes disclosed herein may exhibit multiple substrate specificities and so may, for instance, be able to degrade both PET and oligomers like BHET, MHET, DMT and HEB to TPA, EG and/or carbon dioxide and water.

In some embodiments the polypeptide is capable of degrading polyethylene terephthalate (PET). In some embodiments the polypeptide is also capable of degrading bis(2-hydroxyethyl terephthalate) (BHET) and/or mono(2-hydroxyethyl) terephthalate (MHET). In some embodiments, the polypeptide disclosed herein is capable of degrading low crystallinity (lc) and/or high crystallinity (hc) PET.

As used herein, the terms “crystalline polymer” and “semi-crystalline polymer” are used interchangeably and refer to a polymer in which crystalline regions and amorphous regions coexist. The degree of crystallinity of a semi-crystalline polymer may be estimated by analytical methods known in the art and may range from 10% to 90%. For instance, differential scanning calorimetry (DSC) or X-ray diffraction (e.g., wide-angle X-ray diffraction (WAXD)) may be used to determine the degree of crystallinity of polymers. Other techniques are also suited for estimating a polymer's crystallinity, such as small angle X-ray scattering (SAXS) and infrared spectroscopy.

DSC experiments for determining crystallinity may be conducted as follow: a small quantity of the sample (several mg) is heated at a constant heating rate, from ambient or sub-ambient temperature to a high temperature that is higher than the melting temperature (Tₘ) of the polyester. The heat flow data is collected and plotted against temperature. The degree of crystallinity χ_{c} (%) is calculated as:

$$χ c = \frac{Δ {H}_{f} - Δ {H}_{c c}}{{w}_{t} \times Δ {H}_{f}^{0}} \times 100 \%$$

where ΔH_{f} is the enthalpy of melting, which may be determined by integrating the endothermic melting peak;
ΔH_{cc} is the enthalpy of cold crystallization, which may be determined by integrating the exothermic cold crystallization peak;
wₜ the weight fraction of polymer in the sample; and
ΔH_{f}⁰ is the enthalpy of melting for a fully crystalline polymer, and can be found in literature. As an example, ΔH_{f}⁰ is 140 J/g for 100% crystalline PET (Wunderlich, B., Thermal Analysis, Academic Press, 1990, 417-431).

High crystallinity PET herein refers to a PET material with a crystallinity of at least 37%, e.g., about 37-40%. Low crystallinity PET refers to a PET material with less than 37% crystallinity.

In some embodiments the polypeptide exhibits hydrolase activity at least in a range of temperatures from about 20 °C to about 80 °C. In some embodiments, the polypeptide exhibits hydrolase activity at a temperature between about 20 °C to about 80 °C, between about 25 °C to about 55 °C, between about 25 °C to about 50 °C, between about 30 °C to about 50 °C, between about 35 °C to about 50 °C, between about 40 °C to about 50 °C, between about 40 °C to about 80 °C, between about 50 °C to about 80 °C, or between about 60 °C to about 80 °C.

In some embodiments the polypeptide exhibits hydrolase activity at least in a range of pH from about pH 4 to about pH 10. In some embodiments, the polypeptide exhibits hydrolase activity at a pH between about 4 to about 10, between about pH 5 to about pH 9, between about pH 6 to about pH 8, or between about pH 7 to about pH 8.

In some embodiments the polypeptide is capable of degrading at least 70% of a BHET substrate within 24 hr, or at least 75% of a BHET substrate, at least 80%, at least 85%, at least 90%, or at least 95% of a BHET substrate within 24 hr.

In some embodiments the polypeptide is capable of degrading at least 15% of a PET substrate within 24 hr, or at least 20% of a PET substrate, at least 25%, at least 30%, at least 35%, or at least 40% of a PET substrate within 24 hr. The PET substrate may be a low-crystallinity PET substrate.

In some embodiments the polypeptide is capable of degrading at least 30% of a PET substrate within 48 hr, or at least 35% of a PET substrate, at least 40%, at least 45%, at least 50%, at least 55%, or at least 60% of a PET substrate within 48 hr. The PET substrate may be a low-crystallinity PET substrate.

### Engineered Variants

Disclosed herein is an isolated polypeptide that is distinguished from a wild-type hydrolase enzyme by at least one amino acid substitution, wherein the at least one amino acid substitution is at a position corresponding to position 1, 2, 3, 4, 5, 6, 7, 8, 11, 14, 15, 16, 18, 23, 24, 25, 26, 27, 28, 29, 30, 31, 32, 35, 37, 47, 49, 51, 59, 60, 61, 62, 63, 64, 65, 66, 67, 68, 69, 70, 71, 72, 73, 74, 75, 76, 77, 78, 79, 89, 90, 91, 92, 93, 94, 95, 96, 97, 98, 99, 100, 101, 102, 203, 104, 105, 109, 112, 116, 119, 120, 121, 123, 127, 128, 129, 130, 131, 132, 133, 134, 135, 136, 137, 139, 140, 141, 142, 143, 144, 145, 148, 149, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 193, 201, 202, 205, 206, 207, 208, 209, 210, 211, 212, 213, 214, 215, 216, 217, 218, 219, 220, 221, 222, 223, 224, 225, 226, 227, 228, 231, 232, 236, 239, 240, 245, 246, 247, 248, 249, 250, 251, 252, 253, 257, 258, 261 or 262 of SEQ ID NO: 1-7.

Disclosed herein is an isolated polypeptide that is distinguished from a wild-type hydrolase enzyme by at least one amino acid substitution, wherein the at least one amino acid substitution is at a position corresponding to position 4, 30, 31, 59, 60, 61, 62, 63, 64, 65, 66, 89, 90, 91, 92, 93, 95, 127, 128, 129, 130, 131, 132, 133, 136, 152, 153, 154, 155, 156, 157, 158, 159, 172, 173, 174, 175, 176, 177, 178, 202, 207, 208, 209, 210, 211, 212, 213, 214, 215, 217, 220, 246 or 247 of SEQ ID NO: 1.

Disclosed herein is an isolated polypeptide that is distinguished from a wild-type hydrolase enzyme by at least one amino acid substitution, wherein the at least one amino acid substitution is at a position corresponding to position 24, 35, 37, 43, 44, 62, 63, 64, 65, 66, 67, 68, 69, 70, 92, 93, 94, 95, 96, 97, 131, 132, 133, 134, 135, 136, 137, 155, 156, 157, 158, 159, 160, 161, 162, 175, 176, 177, 178, 179, 180, 181, 182, 206, 211, 212, 213, 214, 215, 216, 217, 218, 219 or 250 of SEQ ID NO: 2.

Disclosed herein is an isolated polypeptide that is distinguished from a wild-type hydrolase enzyme by at least one amino acid substitution, wherein the at least one amino acid substitution is at a position corresponding to position 33, 70, 71, 72, 73, 74, 75, 76, 77, 78, 94, 95, 99, 100, 101, 102, 103, 104, 105, 139, 140, 141, 142, 143, 144, 145, 146, 148, 163, 164, 165, 166, 167, 168, 169, 170, 172, 173, 183, 184, 185, 186, 187, 188, 189, 190, 214, 219, 220, 221, 222, 223, 224, 225, 226, 227, 228 or 258 of SEQ ID NO: 3.

Disclosed herein is an isolated polypeptide that is distinguished from a wild-type hydrolase enzyme by at least one amino acid substitution, wherein the at least one amino acid substitution is at a position corresponding to position 24, 62, 63, 64, 65, 66, 67, 68, 69, 70, 92, 93, 94, 95, 96, 97, 131, 132, 133, 134, 135, 136, 137, 140, 156, 157, 158, 159, 160, 161, 162, 175, 176, 177, 178, 179, 180, 181, 182, 188, 206, 211, 212, 213, 214, 215, 216, 217, 218, 219, 220 or 250 of SEQ ID NO: 4.

Disclosed herein is an isolated polypeptide that is distinguished from a wild-type hydrolase enzyme by at least one amino acid substitution, wherein the at least one amino acid substitution is at a position corresponding to position 15, 23, 25, 31, 32, 64, 90, 92, 109, 120, 136, 140, 154, 160, 163, 172, 173, 180, 191, 201, 202, 207, 210, 236, 245, 246 or 247 of SEQ ID NO: 5.

Disclosed herein is an isolated polypeptide that is distinguished from a wild-type hydrolase enzyme by at least one amino acid substitution, wherein the at least one amino acid substitution is at a position corresponding to position 1, 2, 3, 4, 5, 6, 7, 8, 14, 16, 24, 25, 26, 27, 28, 29, 30, 35, 37, 49, 51, 67, 68, 70, 71, 76, 78, 79, 91, 94, 96, 98, 116, 119, 121, 140, 142, 143, 144, 149, 158, 162, 164, 165, 168, 171, 176, 179, 180, 182, 184, 185, 187, 188, 193, 206, 207, 209, 211, 214, 218, 223, 224, 228, 232, 236, 239, 240, 245, 247, 248, 249, 250, 251, 252, 253, 257, 261 or 262 of SEQ ID NO: 6.

Disclosed herein is an isolated polypeptide that is distinguished from a wild-type hydrolase enzyme by at least one amino acid substitution, wherein the at least one amino acid substitution is at a position corresponding to position 1, 2, 3, 26, 47, 67, 93, 95, 112, 123, 139, 143, 157, 163, 166, 179, 183, 205, 210, 213, 231, 239, 246, 249 or 250 of SEQ ID NO: 7.

In some embodiments the polypeptide comprises an amino acid sequence having at least 70% sequence identity to an amino acid sequence as set forth in SEQ ID NO: 1-7, and further comprises an amino acid substitution at one or more positions corresponding to position 1, 2, 3, 4, 5, 6, 7, 8, 11, 14, 15, 16, 18, 23, 24, 25, 26, 27, 28, 29, 30, 31, 32, 35, 37, 47, 49, 51, 59, 60, 61, 62, 63, 64, 65, 66, 67, 68, 69, 70, 71, 72, 73, 74, 75, 76, 77, 78, 79, 89, 90, 91, 92, 93, 94, 95, 96, 97, 98, 99, 100, 101, 102, 203, 104, 105, 109, 112, 116, 119, 120, 121, 123, 127, 128, 129, 130, 131, 132, 133, 134, 135, 136, 137, 139, 140, 141, 142, 143, 144, 145, 148, 149, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 193, 201, 202, 205, 206, 207, 208, 209, 210, 211, 212, 213, 214, 215, 216, 217, 218, 219, 220, 221, 222, 223, 224, 225, 226, 227, 228, 231, 232, 236, 239, 240, 245, 246, 247, 248, 249, 250, 251, 252, 253, 257, 258, 261 or 262 of SEQ ID NO: 1, SEQ ID NO: 2, SEQ ID NO: 3, SEQ ID NO: 4, SEQ ID NO: 5, SEQ ID NO: 6 or SEQ ID NO: 7.

In some embodiments the polypeptide comprises an amino acid sequence having at least 70% sequence identity to an amino acid sequence as set forth in SEQ ID NO: 1, and further comprises an amino acid substitution at one or more positions corresponding to positions 4, 30, 31, 59, 60, 61, 62, 63, 64, 65, 66, 89, 90, 91, 92, 93, 95, 127, 128, 129, 130, 131, 132, 133, 136, 152, 153, 154, 155, 156, 157, 158, 159, 172, 173, 174, 175, 176, 177, 178, 202, 207, 208, 209, 210, 211, 212, 213, 214, 215, 217, 220, 246 or 247 of SEQ ID NO: 1.

In some embodiments the polypeptide comprises an amino acid sequence having at least 70% sequence identity to an amino acid sequence as set forth in SEQ ID NO: 2, and further comprises an amino acid substitution at one or more positions corresponding to positions 24, 35, 37, 43, 44, 62, 63, 64, 65, 66, 67, 68, 69, 70, 92, 93, 94, 95, 96, 97, 131, 132, 133, 134, 135, 136, 137, 155, 156, 157, 158, 159, 160, 161, 162, 175, 176, 177, 178, 179, 180, 181, 182, 206, 211, 212, 213, 214, 215, 216, 217, 218, 219 or 250 of SEQ ID NO: 2.

In some embodiments the polypeptide comprises an amino acid sequence having at least 70% sequence identity to an amino acid sequence as set forth in SEQ ID NO: 3, and further comprises an amino acid substitution at one or more positions corresponding to positions 33, 70, 71, 72, 73, 74, 75, 76, 77, 78, 94, 95, 99, 100, 101, 102, 103, 104, 105, 139, 140, 141, 142, 143, 144, 145, 146, 148, 163, 164, 165, 166, 167, 168, 169, 170, 172, 173, 183, 184, 185, 186, 187, 188, 189, 190, 214, 219, 220, 221, 222, 223, 224, 225, 226, 227, 228 or 258 of SEQ ID NO: 3.

In some embodiments the polypeptide comprises an amino acid sequence having at least 70% sequence identity to an amino acid sequence as set forth in SEQ ID NO: 4, and further comprises an amino acid substitution at one or more positions corresponding to positions 24, 62, 63, 64, 65, 66, 67, 68, 69, 70, 92, 93, 94, 95, 96, 97, 131, 132, 133, 134, 135, 136, 137, 140, 156, 157, 158, 159, 160, 161, 162, 175, 176, 177, 178, 179, 180, 181, 182, 188, 206, 211, 212, 213, 214, 215, 216, 217, 218, 219, 220 or 250 of SEQ ID NO: 4.

In some embodiments the polypeptide comprises an amino acid sequence having at least 70% sequence identity to an amino acid sequence as set forth in SEQ ID NO: 5, and further comprises an amino acid substitution at one or more positions corresponding to position 15, 23, 25, 31, 32, 64, 90, 92, 109, 120, 136, 140, 154, 160, 163, 172, 173, 180, 191, 201, 202, 207, 210, 236, 245, 246 or 247 of SEQ ID NO: 5.

In some embodiments the polypeptide comprises an amino acid sequence having at least 70% sequence identity to an amino acid sequence as set forth in SEQ ID NO: 6, and further comprises an amino acid substitution at one or more positions corresponding to position 1, 2, 3, 4, 5, 6, 7, 8, 14, 16, 24, 25, 26, 27, 28, 29, 30, 35, 37, 49, 51, 67, 68, 70, 71, 76, 78, 79, 91, 94, 96, 98, 116, 119, 121, 140, 142, 143, 144, 149, 158, 162, 164, 165, 168, 171, 176, 179, 180, 182, 184, 185, 187, 188, 193, 206, 207, 209, 211, 214, 218, 223, 224, 228, 232, 236, 239, 240, 245, 247, 248, 249, 250, 251, 252, 253, 257, 261 or 262 of SEQ ID NO: 6.

In some embodiments the polypeptide comprises an amino acid sequence having at least 70% sequence identity to an amino acid sequence as set forth in SEQ ID NO: 7, and further comprises an amino acid substitution at one or more positions corresponding to position 1, 2, 3, 26, 47, 67, 93, 95, 112, 123, 139, 143, 157, 163, 166, 179, 183, 205, 210, 213, 231, 239, 246, 249 or 250 of SEQ ID NO: 7.

In some embodiments, the polypeptide comprises an amino acid sequence having at least 70% sequence identity to an amino acid sequence as set forth in SEQ ID NO: 1-7, and further comprises an amino acid substitution at one or more positions corresponding to positions 4, 30, 31, 61, 64, 90, 92, 95, 136, 154, 202, 207, 210, 212, 217, 220, 246 or 247 of SEQ ID NO: 1. For example, the polypeptide may comprise 1, 2, 3, 4, 5, 6, 7, 8, 9, 10, 11, 12, 13, 14, 15, 16, 17 or 18 amino acid substitutions at one or more positions corresponding to position 4, 30, 31, 61, 64, 90, 92, 95, 136, 154, 202, 207, 210, 212, 217, 220, 246 or 247 of SEQ ID NO: 1. The polypeptide may comprise at least 1, at least 2, at least 3, at least 4, at least 5, at least 6, at least 7, at least 8, at least 9, at least 10, at least 11, at least 12, at least 13, at least 14, at least 15, at least 16, or at least 17 amino acid substitutions at one or more positions corresponding to position 4, 30, 31, 61, 64, 90, 92, 95, 136, 154, 202, 207, 210, 212, 217, 220, 246, or 247 of SEQ ID NO: 1.

In some embodiments, the polypeptide comprises an amino acid sequence having at least 70% sequence identity to an amino acid sequence as set forth in SEQ ID NO: 1, and further comprises an amino acid substitution at one or more positions corresponding to positions 4, 30, 31, 64, 90, 92, 95, 136, 154, 202, 207, 210, 212, 217, 220, 246 or 247 of SEQ ID NO: 1. For example, the polypeptide may comprise 1, 2, 3, 4, 5, 6, 7, 8, 9, 10, 11, 12, 13, 14, 15, 16, 17 or 18 or amino acid substitutions at one or more positions corresponding to positions 4, 30, 31, 61, 64, 90, 92, 95, 136, 154, 202, 207, 210, 212, 217, 220, 246 or 247 of SEQ ID NO: 1. The polypeptide may comprise at least 1, at least 2, at least 3, at least 4, at least 5, at least 6, at least 7, at least 8, at least 9, at least 10, at least 11, at least 12, at least 13, at least 14, at least 15, at least 16, or at least 17 amino acid substitutions at one or more positions corresponding to positions 4, 30, 31, 61, 64, 90, 92, 95, 136, 154 202, 207, 210, 212, 217, 220, 246 or 247 of SEQ ID NO: 1.

Disclosed herein is an isolated polypeptide that is distinguished from a wild-type hydrolase enzyme by at least one amino acid substitution, wherein the at least one amino acid substitution is at a position corresponding to position 202, 247, 207, 210, 64, 90, 92, 154, 61 or 212 of SEQ ID NO: 1.

In some embodiments, the polypeptide further comprises one or more amino acid substitutions at a position corresponding to position 4, 30, 31, 95, 136, 217, 220 or 246 of SEQ ID NO: 1.

Disclosed herein is an isolated polypeptide that is distinguished from a wild-type hydrolase enzyme by at least one amino acid substitution, wherein the at least one amino acid substitution is at a position corresponding to position 4, 30, 31, 61, 64, 90, 92, 95, 136, 154 202, 207, 210, 212, 217, 220, 246 or 247 of SEQ ID NO: 1.

In some embodiments, the wild-type hydrolase enzyme from which the isolated polypeptide is distinguished comprises an amino acid sequence having at least 70% sequence identity to an amino acid sequence in SEQ ID NO: 1-7.

Disclosed herein is an isolated polypeptide comprising an amino acid sequence that is distinguished from an amino acid sequence of SEQ ID NO: 1-7 by at least one amino acid substitution, wherein the at least one amino acid substitution is at a position corresponding to position 4, 30, 31, 61, 64, 90, 92, 95, 136, 154, 202, 207, 210, 212, 217, 220, 246 or 247 of SEQ ID NO: 1.

In some embodiments, the polypeptide comprises an amino acid substitution at positions corresponding to one of the following sets of positions of SEQ ID NO: 1:
(a) 202 and 247;
(b) 202, 207 and 247;
(c) 202, 210 and 247;
(d) 202, 207, 210 and 247;
(e) 4, 202, 207, 210 and 247;
(f) 30, 202, 207, 210 and 247;
(g) 31, 202, 207, 210 and 247;
(h) 61,202,207, 210 and 247;
(i) 64, 202, 207, 210 and 247;
(j) 90, 202, 207, 210 and 247;
(k) 92, 202, 207, 210 and 247;
(l) 95, 202, 207, 210 and 247;
(m) 136, 202, 207, 210 and 247;
(n) 154, 202, 207, 210 and 247;
(o) 202, 207, 210, 212 and 247;
(p) 202, 207, 210, 217 and 247;
(q) 202, 207, 210, 220 and 247;
(r) 202, 207, 210, 246 and 247;
(s) 64, 90, 92, 136 and 154;
(t) 4, 64, 90, 92, 136 and 154;
(u) 30, 64, 90, 92, 136 and 154;
(v) 31, 64, 90, 92, 136 and 154;
(w) 61, 64, 90, 92, 136 and 154;
(x) 64, 90, 92, 95, 136 and 154;
(y) 64, 90, 92, 136, 154 and 212;
(z) 64, 90, 92, 136, 154 and 217;
(aa) 64, 90, 92, 136, 154 and 220;
(bb) 64, 90, 92, 136, 154 and 246;
(cc) 64, 90, 92, 136, 154, 202 and 247;
(dd) 64, 90, 92, 136, 154, 202, 207 and 247;
(ee) 64, 90, 92, 136, 154, 202, 210 and 247;
(ff) 64, 90, 92, 136, 154, 202, 207, 210 and 247;
(gg) 4, 30, 31, 61, 95, 212, 217, 220 and 246;
(hh) 4, 30, 31, 61, 64, 95, 212, 217, 220 and 246;
(ii) 4, 30, 31, 61, 90, 95, 212, 217, 220 and 246;
(jj) 4, 30, 31, 61, 92, 95, 212, 217, 220 and 246;
(kk) 4, 30, 31, 61, 95, 136, 212, 217, 220 and 246;
(ll) 4, 30, 31, 61, 95, 154, 212, 217, 220 and 246;
(mm) 4, 30, 31, 61, 95, 202, 212, 217, 220, 246 and 247;
(nn) 4, 30, 31, 61, 95, 207, 212, 217, 220 and 246;
(oo) 4, 30, 31, 61, 95, 210, 212, 217, 220 and 246;
(pp) 4, 64, 90, 92, 136, 154, 202, 207, 210 and 247;
(qq) 30, 64, 90, 92, 136, 154, 202, 207, 210 and 247;
(rr) 31, 64, 90, 92, 136, 154, 202, 207, 210 and 247;
(ss) 61, 64, 90, 92, 136, 154, 202, 207, 210 and 247;
(tt) 64, 90, 92, 95, 136, 154, 202, 207, 210 and 247;
(uu) 64, 90, 92, 136, 154, 202, 207, 210, 212 and 247;
(vv) 64, 90, 92, 136, 154, 202, 207, 210, 217 and 247;
(ww) 64, 90, 92, 136, 154, 202, 207, 210, 220 and 247;
(xx) 64, 90, 92, 136, 154, 202, 207, 210, 246 and 247;
(yy) 4, 30, 31, 61, 95, 202, 207, 210, 212, 217, 220, 246 and 247;
(zz) 4, 30, 31, 61, 64, 90, 92, 95, 136, 154, 212, 217, 220 and 246; and
(aaa) 4, 30, 31, 61, 64, 90, 92, 95, 136, 154, 202, 207, 210, 212, 217, 220, 246 and 247.

In some embodiments, the polypeptide comprises one or more amino acid substitutions selected from the following:
(a) a substitution to K, L or I at a position corresponding to position 4 of SEQ ID NO: 1;
(b) a substitution to G, A, V or S at a position corresponding to position 30 of SEQ ID NO: 1;
(c) a substitution to G, A, V, S or T at a position corresponding to position 31 of SEQ ID NO: 1;
(d) a substitution to G, A, V, I or S at a position corresponding to position 61 of SEQ ID NO: 1;
(e) a substitution to G, A, V, I, L or S at a position corresponding to position 64 of SEQ ID NO: 1;
(f) a substitution to M, S, T, F, Y, W, D or E at a position corresponding to position 90 of SEQ ID NO: 1;
(g) a substitution to G, A, V, I, L, S, M, P, F, Y, W or N at a position corresponding to position 92 of SEQ ID NO: 1;
(h) a substitution to G, A, V, I or T at a position corresponding to position 95 of SEQ ID NO: 1;
(i) a substitution to G, A, V, S, H, R or K at a position corresponding to position 136 of SEQ ID NO: 1;
(j) a substitution to G, A, S, T or H at a position corresponding to position 154 of SEQ ID NO: 1;
(k) a substitution to C or A at a position corresponding to position 202 of SEQ ID NO: 1;
(l) a substitution to G, A, V, I, L, S, M, T, P, F, Y or W at a position corresponding to position 207 of SEQ ID NO: 1;
(m) a substitution to G, A, V, I, L, S or M at a position corresponding to position 210 of SEQ ID NO: 1;
(n) a substitution to G, A, V, I, L or S at a position corresponding to position 212 of SEQ ID NO: 1;
(o) a substitution to A, V, I, S or T at a position corresponding to position 217 of SEQ ID NO: 1;
(p) a substitution to V, I, L, S or T at a position corresponding to position 220 of SEQ ID NO: 1;
(q) a substitution to G, A, V or S at a position corresponding to position 246 of SEQ ID NO: 1; and
(r) a substitution to C or A at a position corresponding to position 247 of SEQ ID NO: 1.

In some embodiments, the polypeptide comprises one or more amino acid substitutions selected from the following:
(a) a K substitution at a position corresponding to position 4 of SEQ ID NO: 1;
(b) a A substitution at a position corresponding to position 30 of SEQ ID NO: 1;
(c) a A substitution at a position corresponding to position 31 of SEQ ID NO: 1;
(d) a G substitution at a position corresponding to position 61 of SEQ ID NO: 1;
(e) a A substitution at a position corresponding to position 64 of SEQ ID NO: 1;
(f) a F substitution at a position corresponding to position 90 of SEQ ID NO: 1;
(g) a Y or G substitution at a position corresponding to position 92 of SEQ ID NO: 1;
(h) a A substitution at a position corresponding to position 95 of SEQ ID NO: 1;
(i) a R substitution at a position corresponding to position 136 of SEQ ID NO: 1;
(j) a H substitution at a position corresponding to position 154 of SEQ ID NO: 1;
(k) a C substitution at positions corresponding to positions 202 and 247 of SEQ ID NO: 1;
(l) a I substitution at a position corresponding to position 207 of SEQ ID NO: 1;
(m) a M substitution at a position corresponding to position 210 of SEQ ID NO: 1;
(n) a A substitution at a position corresponding to position 212 of SEQ ID NO: 1;
(o) a A substitution at a position corresponding to position 217 of SEQ ID NO: 1;
(p) a V substitution at a position corresponding to position 220 of SEQ ID NO: 1; and
(q) a A substitution at a position corresponding to position 246 of SEQ ID NO: 1.

The terms “wild-type protein” or “parent protein” are used interchangeably herein and refer to the non-mutated version of a polypeptide as it appears naturally. The terms “mutant”, “variant”, “engineered polypeptide” and “engineered protein” are used interchangeably herein to refer to a polypeptide derived from a wild-type protein and comprising one or more amino acid modifications, e.g., an amino acid substitution, insertion and/or deletion. The variants may be obtained by various techniques well known in the art, e.g., site-directed mutagenesis, random mutagenesis and synthetic oligonucleotide construction.

The term “modification” or “alteration” as used herein in relation to a position in a polypeptide sequence or an amino acid means that the amino acid in the particular position has been modified compared to the amino acid of the wild-type protein.

A “substitution” means that an amino acid residue is replaced by another amino acid residue. An amino acid residue may be replaced by another selected from the naturally-occurring standard 20 amino acid residues, rare naturally occurring amino acid residues (e.g. hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g. cyclohexyl-alanine). Preferably, the term “substitution” refers to the replacement of an amino acid residue by another selected from the naturally-occurring standard 20 amino acid residues (G, P, A, V, L, I, M, C, F, Y, W, H, K, R, Q, N, E, D, S and T). The sign “+” indicates a combination of substitutions. The following terminology is used herein to designate a substitution: N202C denotes that the amino acid residue at position 202 (asparagine, N) of the parent sequence is changed to a cysteine (C). F207V/I denotes that the amino acid residue at position 207 (phenylalanine, F) of the parent sequence is substituted with either a valine (V) or an isoleucine (I).

A “conservative amino acid substitution” is one in which the amino acid residue is replaced with an amino acid residue having a similar side chain. Families of amino acid residues having similar side chains have been defined in the art, which can be generally sub-classified as follows:

a) Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity. Conservative substitutions are shown in the table below under the heading of exemplary substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

b) Exemplary Amino Acid Substitutions

| Original | |
|---|---|
| Residue | Exemplary Substitutions |
| Ala | Val, Leu, Ile |
| Arg | Lys, Gln, Asn |
| Asn | Gln, His, Lys, Arg |
| Asp | Glu |
| Cys | Ser |
| Gln | Asn, His, Lys, |
| Glu | Asp, Lys |
| Gly | Pro |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Ile, Phe |
| Phe | Leu, Val, Ile, Ala |
| Pro | Gly |
| Ser | Thr |
| Thr | Ser |
| Trp | Tyr |
| Tyr | Trp, Phe, Thr, Ser |
| Val | Ile, Leu, Met, Phe, Ala |

The term “deletion”, used in relation to an amino acid, means that the amino acid has been removed or is absent. The term “insertion” means that one or more amino acids have been added.

In some embodiments, the polypeptide comprises an amino acid sequence having at least 70% sequence identity to an amino acid sequence set forth in SEQ ID NO: 1, and one or more amino acid substitutions selected from the following:
(a) a H4K, H4L or H4I substitution at a position corresponding to position 4 of SEQ ID NO: 1;
(b) a L30G, L30A, L30V or L30S substitution at a position corresponding to position 30 of SEQ ID NO: 1;
(c) a L31G, L31A, L31V, L31S or L31T substitution at a position corresponding to position 31 of SEQ ID NO: 1;
(d) a T61G, T61A, T61V, T61I or T61S substitution at a position corresponding to position 61 of SEQ ID NO: 1;
(e) a K64G, K64A, K64V, K64I, K64L or K64S substitution at a position corresponding to position 64 of SEQ ID NO: 1;
(f) a L90M, L90S, L90T, L90F, L90Y, L90W, L90D or L90E substitution at a position corresponding to position 90 of SEQ ID NO: 1;
(g) a Q92G, Q92A, Q92V, Q92I, Q92L, Q92S, Q92M, Q92P, Q92F, Q92Y, Q92W or Q92N substitution at a position corresponding to position 92 of SEQ ID NO: 1;
(h) a S95G, S95A, S95V, S95I or S95T substitution at a position corresponding to position 95 of SEQ ID NO: 1;
(i) a E136G, E136A, E136V, E136S, E136H, E136R or E136K substitution at a position corresponding to position 136 of SEQ ID NO: 1;
(j) a N154G, N154A, N154S, N154T or N154H substitution at a position corresponding to position 154 of SEQ ID NO: 1;
(k) a N202C substitution at a position corresponding to position 202 of SEQ ID NO: 1;
(l) a F207G, F207A, F207V, F207I, F207L, F207S, F207M, F207T, F207P, F207F, F207Y or F207W substitution at a position corresponding to position 207 of SEQ ID NO: 1;
(m) a N210G, N210A, N210V, N210I, N210L, N210S or N210M substitution at a position corresponding to position 210 of SEQ ID NO: 1;
(n) a T212G, T212A, T212V, T212I, T212L or T212S substitution at a position corresponding to position 212 of SEQ ID NO: 1;
(o) a G217A, G217V, G217I, G217S or G217T substitution at a position corresponding to position 217 of SEQ ID NO: 1;
(p) a A220V, A220I, A220L, A220S or A220T substitution at a position corresponding to position 220 of SEQ ID NO: 1;
(q) a L246G, L246A, L246V or L246S substitution at a position corresponding to position 246 of SEQ ID NO: 1; and
(r) a T247C substitution at a position corresponding to position 247 of SEQ ID NO: 1.

In some embodiments, the polypeptide comprises an amino acid sequence having at least 70% sequence identity to an amino acid sequence set forth in SEQ ID NO: 1, and one or more amino acid substitutions selected from the following:
(a) a H4K substitution at a position corresponding to position 4 of SEQ ID NO: 1;
(b) a L30A substitution at a position corresponding to position 30 of SEQ ID NO: 1;
(c) a L31A substitution at a position corresponding to position 31 of SEQ ID NO: 1;
(d) a T61G substitution at a position corresponding to position 61 of SEQ ID NO: 1;
(e) a K64A substitution at a position corresponding to position 64 of SEQ ID NO: 1;
(f) a L90F substitution at a position corresponding to position 90 of SEQ ID NO: 1;
(g) a Q92Y or Q92G substitution at a position corresponding to position 92 of SEQ ID NO: 1;
(h) a S95A substitution at a position corresponding to position 95 of SEQ ID NO: 1;
(i) a E136R substitution at a position corresponding to position 136 of SEQ ID NO: 1;
(j) a N154H substitution at a position corresponding to position 154 of SEQ ID NO: 1;
(k) a N202C substitution at a position corresponding to position 202 of SEQ ID NO: 1;
(1) a F207I substitution at a position corresponding to position 207 of SEQ ID NO: 1;
(m) a N210M substitution at a position corresponding to position 210 of SEQ ID NO: 1;
(n) a T212A substitution at a position corresponding to position 212 of SEQ ID NO: 1;
(o) a G217A substitution at a position corresponding to position 217 of SEQ ID NO: 1;
(p) a A220V substitution at a position corresponding to position 220 of SEQ ID NO: 1;
(q) a L246A substitution at a position corresponding to position 246 of SEQ ID NO: 1; and
(r) a T247C substitution at a position corresponding to position 247 of SEQ ID NO: 1;

In some embodiments, the polypeptide comprises one or more amino acid substitutions in mutation group 1.

### Mutation Group 1:

(a) a C substitution at positions corresponding to positions 202 and 247 of SEQ ID NO: 1;
(b) a I substitution at a position corresponding to position 207 of SEQ ID NO: 1; and
(c) a M substitution at a position corresponding to position 210 of SEQ ID NO: 1.

In some embodiments, the polypeptide comprises one or more amino acid substitutions in mutation group 2.

### Mutation Group 2:

(a) a A substitution at a position corresponding to position 64 of SEQ ID NO: 1;
(b) a F substitution at a position corresponding to position 90 of SEQ ID NO: 1;
(c) a Y or G substitution at a position corresponding to position 92 of SEQ ID NO: 1;
(d) a R substitution at a position corresponding to position 136 of SEQ ID NO: 1; and
(e) a H substitution at a position corresponding to position 154 of SEQ ID NO: 1.

In some embodiments, the polypeptide comprises one or more amino acid substitutions in mutation group 3.

### Mutation Group 3:

(a) a K substitution at a position corresponding to position 4 of SEQ ID NO: 1;
(b) a A substitution at a position corresponding to position 30 of SEQ ID NO: 1;
(c) a A substitution at a position corresponding to position 31 of SEQ ID NO: 1;
(d) a G substitution at a position corresponding to position 61 of SEQ ID NO: 1;
(e) a A substitution at a position corresponding to position 95 of SEQ ID NO: 1;
(f) a A substitution at a position corresponding to position 212 of SEQ ID NO: 1;
(g) a A substitution at a position corresponding to position 217 of SEQ ID NO: 1;
(h) a V substitution at a position corresponding to position 220 of SEQ ID NO: 1; and
(i) a A substitution at a position corresponding to position 246 of SEQ ID NO: 1.

In some embodiments, the polypeptide comprises one or more amino acid substitutions in each of mutation groups 1 and 2. In some embodiments, the polypeptide comprises one or more amino acid substitutions in each of mutation groups 1 and 3. In some embodiments, the polypeptide comprises one or more amino acid substitutions in each of mutation groups 2 and 3. In some embodiments, the polypeptide comprises one or more amino acid substitutions in each of mutation groups 1, 2 and 3.

In some embodiments, the polypeptide comprises all of the amino acid substitutions in mutation group 1. In some embodiments, the polypeptide comprises all of the amino acid substitutions in mutation group 1 and one or more amino acid substitutions in mutation group 2. In some embodiments, the polypeptide comprises all of the amino acid substitutions in mutation group 1 and one or more amino acid substitutions in mutation group 3. In some embodiments, the polypeptide comprises all of the amino acid substitutions in mutation group 1 and one or more amino acid substitutions in each of mutation groups 2 and 3. In some embodiments, the polypeptide comprises all of the amino acid substitutions in mutation groups 1 and 2. In some embodiments, the polypeptide comprises all of the amino acid substitutions in mutation groups 1 and 3. In some embodiments, the polypeptide comprises all of the amino acid substitutions in mutation groups 1, 2 and 3.

In some embodiments, the polypeptide comprises all of the amino acid substitutions in mutation group 2. In some embodiments, the polypeptide comprises all of the amino acid substitutions in mutation group 2 and one or more amino acid substitutions in mutation group 1. In some embodiments, the polypeptide comprises all of the amino acid substitutions in mutation group 2 and one or more amino acid substitutions in mutation group 3. In some embodiments, the polypeptide comprises all of the amino acid substitutions in mutation group 2 and one or more amino acid substitutions in each of mutation groups 1 and 3. In some embodiments, the polypeptide comprises all of the amino acid substitutions in mutation groups 2 and 3.

In some embodiments, the polypeptide comprises all of the amino acid substitutions in mutation group 3. In some embodiments, the polypeptide comprises all of the amino acid substitutions in mutation group 3 and one or more amino acid substitutions in mutation group 1. In some embodiments, the polypeptide comprises all of the amino acid substitutions in mutation group 3 and one or more amino acid substitutions in mutation group 2. In some embodiments, the polypeptide comprises all of the amino acid substitutions in mutation group 3 and one or more amino acid substitutions in each of mutation groups 1 and 2.

In some embodiments, the polypeptide comprises all of the amino acid substitutions in mutation groups 1 and 2, and one or more amino acid substitutions in mutation group 3. In some embodiments, the polypeptide comprises all of the amino acid substitutions in mutation groups 1 and 3, and one or more amino acid substitutions in mutation group 2. In some embodiments, the polypeptide comprises all of the amino acid substitutions in mutation groups 2 and 3, and one or more amino acid substitutions in mutation group 1.

In some embodiments, the polypeptide comprises an amino acid sequence having at least 70% sequence identity to an amino acid sequence set forth in SEQ ID NO: 1, and one of the following sets of substitutions:
(a) N202C and T247C;
(b) N202C, F207I and T247C;
(c) N202C, N210M and T247C;
(d) N202C, F207I, N210M and T247C;
(e) H4K, N202C, F207I, N210M and T247C;
(f) L30A, N202C, F207I, N210M and T247C;
(g) L31A, N202C, F207I, N210M and T247C;
(h) T61G, N202C, F207I, N210M and T247C;
(i) K64A, N202C, F207I, N210M and T247C;
(j) L90F, N202C, F207I, N210M and T247C;
(k) Q92Y/G, N202C, F207I, N210M and T247C;
(l) S95A, N202C, F207I, N210M and T247C;
(m) E136R, N202C, F207I, N210M and T247C;
(n) N154H, N202C, F207I, N210M and T247C;
(o) N202C, F207I, N210M, T212A and T247C;
(p) N202C, F207I, N210M, G217A and T247C;
(q) N202C, F207I, N210M, A220V and T247C;
(r) N202C, F207I, N210M, L246A and T247C;
(s) K64A, L90F, Q92Y/G, E136R and N154H;
(t) H4K, K64A, L90F, Q92Y/G, E136R and N154H;
(u) L30A, K64A, L90F, Q92Y/G, E136R and N154H;
(v) L31A, K64A, L90F, Q92Y/G, E136R and N154H;
(w) T61G, K64A, L90F, Q92Y/G, E136R and N154H;
(x) K64A, L90F, Q92Y/G, S95A, E136R and N154H;
(y) K64A, L90F, Q92Y/G, E136R, N154H and T212A;
(z) K64A, L90F, Q92Y/G, E136R, N154H and G217A;
(aa) K64A, L90F, Q92Y/G, E136R, N154H and A220V;
(bb) K64A, L90F, Q92Y/G, E136R, N154H and L246A;
(cc) K64A, L90F, Q92Y/G, E136R, N154H, N202C and T247C;
(dd) K64A, L90F, Q92Y/G, E136R, N154H, N202C, F207I, and T247C;
(ee) K64A, L90F, Q92Y/G, E136R, N154H, N202C, N210M and T247C;
(ff) K64A, L90F, Q92Y/G, E136R, N154H, N202C, F207I, N210M and T247C;
(gg) H4K, L30A, L31A, T61G, S95A, T212A, G217A, A220V and L246A;
(hh) H4K, L30A, L31A, T61G, K64A, S95A, T212A, G217A, A220V and L246A;
(ii) H4K, L30A, L31A, T61G, L90F, S95A, T212A, G217A, A220V and L246A;
(jj) H4K, L30A, L31A, T61G, Q92Y/G, S95A, T212A, G217A, A220V and L246A;
(kk) H4K, L30A, L31A, T61G, S95A, E136R, T212A, G217A, A220V and L246A;
(ll) H4K, L30A, L31A, T61G, S95A, N154H, T212A, G217A, A220V and L246A;
(mm) H4K, L30A, L31A, T61G, S95A, N202C, T212A, G217A, A220V, L246A and T247C;
(nn) H4K, L30A, L31A, T61G, S95A, F207I, T212A, G217A, A220V and L246A;
(oo) H4K, L30A, L31A, T61G, S95A, N210M, T212A, G217A, A220V and L246A;
(pp) H4K, K64A, L90F, Q92Y/G, E136R, N154H, N202C, F207I, N210M and T247C;
(qq) L30A, K64A, L90F, Q92Y/G, E136R, N154H, N202C, F207I, N210M and T247C;
(rr) L31A, K64A, L90F, Q92Y/G, E136R, N154H, N202C, F207I, N210M and T247C;
(ss) T61G, K64A, L90F, Q92Y/G, E136R, N154H, N202C, F207I, N210M and T247C;
(tt) K64A, L90F, Q92Y/G, S95A, E136R, N154H, N202C, F207I, N210M and T247C;
(uu) K64A, L90F, Q92Y/G, E136R, N154H, N202C, F207I, N210M, T212A and T247C;
(vv) K64A, L90F, Q92Y/G, E136R, N154H, N202C, F207I, N210M, G217A and T247C;
(ww) K64A, L90F, Q92Y/G, E136R, N154H, N202C, F207I, N210M, A220V and T247C;
(xx) K64A, L90F, Q92Y/G, E136R, N154H, N202C, F207I, N210M, L246A and T247C
(yy) H4K, L30A, L31A, T61G, S95A, N202C, F207I, N210M, T212A, G217A, A220V, L246A and T247C;
(zz) H4K, L30A, L31A, T61G, K64A, L90F, Q92Y/G, S95A, E136R, N154H, T212A, G217A, A220V and L246A; and
(aaa) H4K, L30A, L31A, T61G, K64A, L90F, Q92Y/G, S95A, E136R, N154H, N202C, F207I, N210M, T212A, G217A, A220V, L246A and T247C.

Disclosed herein is the polypeptide comprising an amino acid sequence having at least 70% sequence identity to an amino acid sequence set forth in 14-30. In some embodiments the polypeptide comprises an amino acid having at least 70%, at least 75%, at least 80%, at least 85%, at least 90%, at least 91%, at least 92%, at least 93%, at least 94%, at least 95%, at least 96%, at least 97%, at least 98% or at least 99% sequence identity to an amino acid sequence set forth in SEQ ID NO: 14-30.

The one or more amino acid substitutions in the polypeptide may improve one or more properties of the polypeptide, including but not limited to the thermostability of the polypeptide, the enzymatic activity of the polypeptide, and the range of substrates and/or binding partners recognized by the polypeptide.

In some embodiments the polypeptide variant exhibits an increased ability to degrade an ester-containing product or material, more particularly a polyester-containing product or material, as compared to a polypeptide of SEQ ID NO: 1-7. Such an increase is typically of about 5%, 10%, 20%, 30%, 40%, 50%, 100%, 200%, 300%, 400%, 500% or more in comparison to the parent polypeptide.

The ester degrading activity of a polypeptide may be evaluated by one skilled in the art according to methods known in the art. For instance, the activity can be assessed by the measurement of a specific ester's degradation rate, the measurement of the decrease of the turbidity of an emulsion containing a ester compound, the measurement of the amount of an ester's breakdown products using mass spectrometry, the measurement of the loss of mass from an ester-containing product or material, etc.

As used herein, the term “specific degrading activity” for a targeted polyester designates the initial rate of monomers and/or oligomers, in mass units (e.g., mg), released per hour and per mg of enzyme under suitable conditions of temperature, pH and buffer, when contacting a product containing said targeted polyester with a polypeptide disclosed herein. As an example, the specific degrading activity for PET corresponds to the mass of MHET, BHET and TPA produced per day and per mg of polypeptide.

In some embodiments a polypeptide variant exhibits measurable hydrolase activity at least in a range of temperatures from 10 °C to 90 °C. In some embodiments a polypeptide variant exhibits measurable hydrolase activity at a temperature between about 20 °C to about 90 °C, about 25 °C to about 85 °C, about 30 °C to about 80 °C, about 30 °C to about 70 °C, about 30 °C to about 60 °C, about 35 °C to about 55 °C, between about 40 °C to about 50 °C, between about 40 °C to about 80 °C, between about 50 °C to about 80 °C, or between about 60 °C to about 80 °C.

In some embodiments a polypeptide variant exhibits hydrolase activity at least 5% higher, at least 10%, 20%, 30%, 40%, 50%, 60%, 70%, 80%, 90%, 100%, 125%, 150%, 175%, 200%, 250%, or at least 300% higher than the hydrolase activity of the polypeptide of SEQ ID NO: 1-6 over a period of at least 48 hr.

In some embodiments a polypeptide variant exhibits hydrolase activity at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 15-fold, at least 16-fold, at least 17-fold, at least 18-fold, at least 19-fold, or at least 20-fold higher than the hydrolase activity of the polypeptide of SEQ ID NO: 1-7 over a period of at least 48 hr.

In some embodiments, a polypeptide variant exhibits a measurable hydrolase activity at least in a range of pH from 4 to 11. In some embodiments, a polypeptide variant exhibits a measurable hydrolase activity in a range of pH from 4 to 10, from 5 to 9, from 6 to 8, or from 7 to 8.

In some embodiments, a polypeptide variant exhibits improved thermostability compared to the thermostability of the polypeptide of SEQ ID NO: 1-7. As used herein, the term “improved thermostability” indicates an increased ability of a polypeptide to resist changes in its chemical and/or physical structure at elevated temperatures, and more particularly at temperatures between 30 °C and 90 °C, as compared to a polypeptide of SEQ ID NO: 1-7.

In some embodiments, a polypeptide variant exhibits a higher or equivalent melting temperature (Tₘ) as compared to the polypeptide of SEQ ID NO: 1-7. As used herein, the “melting temperature (Tₘ)” of a given protein corresponds to the temperature at which 50% of said protein is denatured. Circular dichroism (CD) may be used to quantify the change in thermal denaturation temperature of a protein and thereby to determine its melting temperature (Tₘ).

The thermostability of a protein may be evaluated by one skilled in the art according to methods known in the art. For instance, thermostability can be assessed by measuring the residual polypeptide activity or the residual polyester hydrolase activity after incubation at different temperatures. The ability to perform multiple rounds of hydrolytic assays at different temperatures can also be evaluated. Alternatively, or in addition, differential scanning fluorimetry (DSF) may be performed to assess the thermostability of a polypeptide or hydrolase.

### Enzyme Production

Disclosed herein is a polynucleotide encoding a polypeptide as defined herein.

Disclosed herein is an expression vector comprising a polynucleotide as defined herein.

The term “polynucleotide” or “nucleic acid” is used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA. Polynucleotides can be isolated from natural sources, synthesised in vitro, or prepared from a combination of natural and synthetic molecules using methods known in the art.

The codon usage in a polynucleotide disclosed herein may be adapted according to the host cell in which the nucleic acids will be transcribed. These steps may be carried out according to methods well known to one skilled in the art. A polynucleotide herein may further comprise additional nucleotide sequences, such as regulatory regions, i.e., promoters, enhancers, silencers, terminators, signal peptides, secretion peptides and the like that can be used to cause or regulate expression of the polypeptide in a selected host cell or system.

The term “expression”, as used herein, refers to any step involved in the production of a polypeptide including, but being not limited to, transcription, post-transcriptional modification, translation, post-translational modification, and secretion.

The term “expression cassette” denotes a polynucleotide comprising a coding region, i.e., a polynucleotide encoding a polypeptide as defined herein, and a regulatory region, i.e., comprising one or more control sequences, operably linked. The regulatory region may comprise a promoter, e.g., a transcriptional promoter or transcription terminator. The regulatory region may include a promoter that is recognised by a host cell or an in vitro expression system for expression of a nucleic acid encoding a polypeptide as defined herein. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The regulatory region may also contain a transcription terminator, which is recognised by a host cell to terminate transcription. The terminator is operably linked to the 3′-terminus of the nucleic acid encoding the protease. Any terminator that is functional in the host cell may be used in the present invention. Typically, an expression cassette comprises, or consists of, a polynucleotide as defined herein operably linked to a transcriptional promoter and a transcription terminator.

By “expression vector” or “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

Disclosed herein is a host cell comprising a polynucleotide, expression cassette or expression vector as defined herein.

The host cell may be transformed, transfected or transduced with the polynucleotide, expression cassette or expression vector in a transient or stable manner. The expression cassette or vector disclosed herein may be maintained in the host cell as a chromosomal integrant or as a self-replicating extra-chromosomal vector. The term “host cell” also encompasses any progeny of a parent host cell that is not identical to the parent host cell due to mutations that occur during replication. The host cell may be any cell useful in the production of a polypeptide or polypeptide variant disclosed herein, e.g., a prokaryote or a eukaryote. The prokaryotic host cell may be any Gram-positive or Gram-negative bacterium. The host cell may also be a eukaryotic cell, such as a yeast, fungal, mammalian, insect or plant cell.

The polynucleotide, expression cassette or expression vector disclosed herein may be introduced into the host cell by any method known by the skilled person, such as electroporation, conjugation, transduction, competent cell transformation, protoplast transformation, protoplast fusion, biolistic “gene gun” transformation, PEG-mediated transformation, lipid-assisted transformation or transfection, chemically mediated transfection, lithium acetate-mediated transformation, liposome-mediated transformation. More than one copy of a polynucleotide, cassette or vector disclosed herein may be inserted into a host cell to increase production of a polypeptide or polypeptide variant disclosed herein. The host cell may be engineered to have an improved capacity to degrade ester containing material. For instance, an amino acid sequence disclosed herein may be used to complement a wild-type strain of fungus or bacterium already known as able to degrade esters or polyesters, in order to improve and/or increase the strain capacity.

In some embodiments the host cell is selected from the group consisting of Gram-positive bacteria, Gram-negative bacteria, filamentous fungi, yeast and algae. Non-limiting examples of host cells include Escherichia coli, Lactococcus lactis, Bacillus sp., Pichia sp., Streptomyces sp. and Actinobacteria.

In some embodiments the host cell is a bacterial strain from Actinobacteria. In some embodiments the host cell is a bacterial strain from the Streptomyces genus. In some embodiments the host cell is a bacterial strain from the Microbispora, Nonomuraea or Micromonospora genus. In one embodiment the host cell is Microbispora hainanensis, Microbispora camponoti, Nonomuraea soli, Nonomuraea sp. TT08I-71, Micromonospora chersina or Micromonospora musae.

Disclosed herein is a method of producing a polypeptide as defined herein, comprising culturing a host cell defined herein under conditions suitable for expressing the polypeptide.

In some embodiments the polypeptide is secreted extracellularly. In some embodiments the polypeptide is recovered from the cell culture.

The host cells may be cultivated by methods well known in art, e.g., by shake flask cultivation or small-scale or large-scale fermentation (including continuous, batch, fed-batch, or solid state fermentations) in laboratory or industrial fermentors performed in a suitable medium and under conditions allowing the polypeptide to be expressed and/or isolated.

Where the polypeptide is secreted extracellularly into the nutrient medium, the protease can be recovered directly from the culture supernatant. Conversely, the polypeptide can be recovered from cell lysates or after permeabilisation. The polypeptide may be recovered using any method known in the art. For example, the polypeptide may be recovered from the nutrient medium by conventional procedures including, but not limited to, collection, centrifugation, filtration, extraction, spray-drying, evaporation, or precipitation. Alternatively, the polypeptide may be partially or totally purified by a variety of procedures known in the art including, but not limited to, chromatography (e.g., ion exchange, affinity, hydrophobic and size exclusion), electrophoretic procedures (e.g., preparative isoelectric focusing), differential solubility (e.g., ammonium sulfate precipitation), SDS-PAGE, or extraction to obtain substantially pure polypeptides.

### Polyester Degradation

Disclosed herein is a method of degrading a product comprising at least one polyester, the method comprising contacting the product with a polypeptide defined herein and/or with a cell expressing a polypeptide defined herein, under conditions suitable for degrading the at least one polyester.

In some embodiments the polyester is selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxyalkanoate (PHA), polyethylene succinate (PES), polybutylene succinate (PBS), polybutylene succinate adipate (PBSA), polybutylene adipate terephthalate (PBAT), polyethylene furanoate (PEF), polycaprolactone (PCL), polyethylene adipate (PEA) and polyethylene naphthalate (PEN), polycyclohexylenedimethylene terephthalate (PCT), and co-polymers thereof. The product may comprise a combination of two or more different polyesters. In one embodiment the polyester is PET. The PET may be, for example, low crystallinity PET (lcPET) and/or high crystallinity PET (hcPET).

As used herein, a “material” or “product” containing a polyester may be an article or product in any shape, construction or state, such as a plastic sheet, tube, rod, container, fibre, textile, solid, semi-solid, liquid, suspension, emulsion, etc. Preferably, the material or product is a manufactured article, such as rigid or flexible packaging, films, bags and sacks, electrical insulation, filters, carpet scrap, fabrics, textiles, etc. The material or product may contain additional substances or additives, such as plasticizers, minerals, organic fillers or dyes. The material or product may comprise a mix of semi-crystalline and/or amorphous polyesters and/or additives.

A material or product for polyester degradation herein may have undergone a prior physical, chemical and/or biological processing step, e.g., mechanical fragmentation to increase surface area for degradation, chemical treatment to reduce crystallinity or remove additives that may poison the cell or polypeptide, or disinfection to remove contaminating organisms. In some embodiments, the product is transformed into an emulsion or a powder, which is added to a liquid medium containing the cell and/or polypeptide. The product may also undergo ozonation to modify its structure, e.g., to reduce its crystallinity. Alternatively or additionally, a thermal or ultraviolet pre-treatment can be applied (e.g., using microwaves) to disinfect, pasteurise or sterilise of the plastic product. The product may also be sorted, washed, disinfected and/or sterilised prior to ester degradation. Several pre-treatments may be combined.

The method may comprise contacting the product with a polypeptide defined herein, such as a wild-type of engineered polypeptide as defined herein. In some embodiments, the polypeptide comprises an amino acid sequence having at least 70% sequence identity to an amino acid sequence of SEQ ID NO: 1-30. The polypeptide may be used in purified form, either alone or in combination with additional polypeptides or enzymes, for ester degradation. The polypeptide may be in soluble form, or in solid phase such as a powder. The polypeptide may be bound to cell membranes or lipid vesicles, or to synthetic supports such as glass, plastic, polymers, filters, membranes, e.g., in the form of beads, columns, plates and the like. Alternatively, culture preparations containing the polypeptide may be used for ester degradation. For example a lyophilised culture medium of a microorganism expressing and secreting the polypeptide may be used.

The method may comprise contacting the product with a cell expressing a polypeptide defined herein. The cell may be secreting the polypeptide to the culture medium or towards the cell membrane of the microorganism wherein said polypeptide may be anchored. The cell may naturally synthesise the polypeptide, or it may be a recombinant cell comprising a polynucleotide encoding the polypeptide.

Several types of cells and/or polypeptide preparations may be used together or sequentially to improve degradation of products containing different polyesters.

The conditions for degradation may vary depending on the nature of the product and its polyester component (i.e., the shape of the product and its composition, whether the product has undergone pre-processing, the molecular weight of the polyester, etc.); the polypeptide or cell used (including the amount used); and various process parameters (e.g., temperature, pH, additional agents, etc.). Generally, the temperature is maintained below an inactivating temperature, which corresponds to the temperature at which the polypeptide is inactivated and/or the cell no longer synthesises the polypeptide. Further, the pH is maintained at the optimal pH for polypeptide or cell activity. One skilled in the art may easily adapt the method parameters to a given product or polyester and/or to the polypeptide or cell.

In some embodiments of the method, the product is in contact with the cell or the polypeptide at a pH of about 6 to about 8. In one embodiment, the product is in contact with the cell or the polypeptide at a pH of about 7.

In some embodiments of the method, the product is in contact with the cell or polypeptide at a temperature of about 25 °C to about 50 °C. The product may be in contact with the cell or polypeptide at a temperature of about 25 °C to about 50 °C, about 30 °C to about 50 °C, about 35 °C to about 50 °C, about 40 °C to about 50 °C, or about 45 °C to about 50 °C. The product may be in contact with the cell or polypeptide at a temperature of about 25 °C, about 30 °C, about 35 °C, about 40 °C, about 45 °C, or about 50 °C.

In some embodiments of the method, the product is in contact with the cell or polypeptide for at least 24 hr. The product may be in contact with the cell or polypeptide for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days. The length of contact between the product and polypeptide or cell may depend on the nature of the product and its polyester component, the polypeptide or cell used, the process parameters, and the extent of degradation desired.

Optionally, monomers and/or oligomers resulting from the degradation may be recovered, sequentially or continuously. A single type of monomer and/or oligomer or several different types of monomers and/or oligomers may be recovered, depending on the starting polyester-containing product. The recovered monomers and/or oligomers may be used for polyester synthesis.

Disclosed herein is a kit comprising a polypeptide as defined herein. The polypeptide may be a wild-type or engineered hydrolase. The kit may be used for degrading a polyester-containing product or material, e.g., a product or material containing PET. The polypeptide may be in soluble form, or in solid phase such as a powder. The polypeptide may be bound to a biological substrate, such as a cell membrane or lipid vesicle, or to synthetic supports such as glass, plastic, polymers, filters, membranes, e.g., in the form of beads, columns, plates and the like.

Provided herein is a composition comprising a polypeptide as defined herein. The composition may comprise one or more additional proteins (such as one or more other hydrolases or enzymes), excipients or additives, e.g., glycerol, sorbitol or dextrin (such as maltodextrine and/or cyclodextrine), starch, glycol (such as propanediol), and/or salt, etc.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications, which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Materials and Methods

All solvents used in product analysis (acetonitrile, formic acid, dimethylformamide) were purchased from commercial suppliers. Bis(2-hydroxyethyl) terephthalate (BHET), terephthalic acid (TPA), potassium phosphate (NaH₂PO₄ and Na₂HPO₄), and 4-bromobenzoic acid were obtained from Sigma-Aldrich. High crystallinity PET film (ES301250) and low crystallinity PET film (ES301445) were obtained from Goodfellow and was ground into powder.

### Plasmid Construction

Codon-optimized DNA sequences obtained from Escherichia coli (E. coli) were synthesized from Twist Biosciences. These inserts were assembled via golden gate into pET28a(+), cloned with E. coli Omnimax competent cells. E. coli XJb (DE3) autolysis competent cells (Zymo Research) was subsequently used for the transformation of these hexahistidine-tagged constructs for protein expression. Tables 4 and 5 show the amino acid sequences of the gene used and the solubility tags used.

### High Throughput Screening

1 mL overnight autoinduction media (Merck) was used for the inoculation of single colonies obtained from the transformation, in which 50 μg/mL kanamycin, 1.5 M L-arabinose and 0.5 M magnesium chloride was also added. A single freeze thaw cycle in lysis buffer (50 mM sodium phosphate buffer, 300 mM sodium chloride, 10 mM imidazole and 0.03% Triton X-100), from the cell pellet harvested allowed the cell to release its embedded protein. To capture these His-tagged proteins, Ni resin (Pure Cube) was used. These proteins were subsequently eluted in 50 mM sodium phosphate buffer pH 7.0, 300 mM sodium chloride and 500 mM imidazole. Based on the protein gel obtained, ImageJ was used to assess protein elutes for solubility—where the intensity of each band obtained was normalized to the intensity of the reference band on the standard ladder used, as well as molecular weight of each protein. Based on this normalized intensity obtained, the best expressing constructs were scaled up for purification and subsequent characterization.

### 1 L Culture Purification

A starter culture grown in LB Broth (Miller) containing 50 μg/mL kanamycin was prepared, where a single colony from the transformation was inoculated overnight at 37 °C. The starter culture was diluted 200× in a fresh 1 L LB Broth containing 50 μg/mL kanamycin, 1.5 M L-arabinose and 0.5 M Magnesium chloride. Subcultures were grown at 37 °C until the optical density at 600 nm (OD600) reached ~0.4-0.5. 100 μM isopropyl β-D-1-thiogalactopyranoside (IPTG) was then added to induce the expression of proteins. Following which, the cultures were incubated overnight at 16 °C for 18 to 20 hours. Cells were harvested from this culture using a centrifuge maintained at 4 °C, before resuspending and freezing the resulting pellet at −80 °C in 10 mL of lysis buffer containing 50 mM sodium phosphate buffer pH 7.0, 300 mM sodium chloride, 10 mM imidazole and 0.03% Triton X-100. To ensure protein stability, subsequent purification steps were carried out at 4 °C.

The frozen pellet was thawed with the addition of 10 mL lysis buffer and sonicated to release proteins from the cells. The supernatant obtained from centrifugation of lysates at 13,500×g was loaded on Ni resin and incubated for 1 hour to capture the His-tagged proteins. 20 mL of 50 mM sodium phosphate buffer, 300 mM sodium chloride and 50 mM imidazole was then used to wash the resin, before eluting the bound proteins with 50 mM sodium phosphate buffer pH 7.0, 300 mM sodium chloride and 500 mM imidazole. Lastly, the proteins were exchanged into 50 mM sodium phosphate pH 7.0 with 10% glycerol to allow for long-term storage at −80 °C. Subsequent BHET and PET degradation studies were carried out with these purified proteins.

### BHET Depolymerisation Assay

BHET stock solution (1 M) was made by dissolving in dimethylformamide. BHET stock solution (2.5 μL, 5 mM) was pipetted into a 2 mL glass vial containing 500 μL of 100 mM potassium phosphate buffer (pH 7) and 1.67 μM purified protein. The glass vial was tightly capped, and the reaction mixture was incubated at 25 °C for 24 hours in an Eppendorf ThermoMixer®. After 24 hours, the reaction is quenched with 500 μL of methanol. The mixture was transferred into a 10 mL centrifuge tube, the vial was washed with 4 mL of buffer/methanol (1:1, v/v) and the contents were transferred to the centrifuge tube. This is followed by the addition of 5 mL 0.5 mM 4-bromobenzoic acid in buffer/methanol (1:1, v/v) as an internal standard. The reaction mixture was sonicated, and an aliquot was filtered with a 0.2 μm syringe filter and analyzed via UHPLC-MS.

### Calibration of BHET and TPA

BHET and TPA stock solutions (1 mM) were prepared by dissolving the solids in 5 mL of in buffer/methanol (1:1, v/v), followed by the addition of 5 mL 0.5 mM 4-bromobenzoic acid in buffer/methanol (1:1, v/v) as an internal standard. 6 concentrations ranging from 0.05 mM to 1 mM were prepared from the stock solution and analyzed via UPHLC-MS. A calibration was plotted with the molar ratio against area ratio.

### Calibration of MHET

MHET (1 M) was prepared by dissolving the solids in dimethylformamide. Six concentrations ranging from 0.03 mM to 0.18 mM were prepared from the MHET stock solution, topped up with 0.5 mM 4-bromobenzoic acid internal standard and analyzed via UPHLC-MS. A calibration was plotted with the molar ratio against area ratio.

### PET Depolymerisation Assay

PET powder (2 mg, 20 mM) was weighed into a 2 mL glass vial and fully submerged in 500 μL of 100 mM potassium phosphate buffer (pH 7 or 8) with 1.67 μM purified protein. The glass vial was tightly capped, and the reaction mixture was incubated at 500 rpm and 25-70 °C for 3 hours to up to 28 days in an Eppendorf ThermoMixer®. The reaction is quenched with 500 μL of methanol. The mixture was transferred into a 10 mL centrifuge tube, the vial was washed with 4 mL of buffer/methanol (1:1, v/v) and the contents were transferred to the centrifuge tube. This is followed by the addition of 5 mL 0.5 mM 4-bromobenzoic acid in buffer/methanol (1:1, v/v) as an internal standard. The reaction mixture was sonicated, and an aliquot was filtered with a 0.2 μm syringe filter and analyzed via UHPLC-MS.

### EFI-EST Tool for Generating SSN

The online EFI-Enzyme Similarity Tool offered by Illinois Carl R. Woese Institute for Genomic Biology was used to generate the SSN for SIBER 1. The amino acid sequence of SIBER 1 (SEQ ID NO: 1) was used as the query sequence to generate an SSN for a single protein against its closest homologues in the UniProt database. An all-by-all BLAST was performed to obtain the similarities between each sequence pair to calculate edge values to generate the SSN. The BLAST retrieval options selected were the default values of 5 E-value and 1000 maximum number of retrieved sequences. SSN edge calculation E-value used was the default value of 5. After SSN generation, the alignment score of 63 was selected as the threshold value to filter the nodes and obtain a total node count of 4284. The filtered SSN was downloaded and visualized using the software Cytoscape.

### Cytoscape SSN Visualization

Within Cytoscape, yFiles Organic Layout was selected as the SSN display layout. Stray, un-clustered proteins were removed from the SSN to remove clutter and increase resolution to obtain the final node count of 2006 nodes. Non-prokaryotic proteins were highlighted by searching the SSN using the superkingdom node-filter of “Eukaryote”. The remaining bacterial proteins were then color-coded according to their phylum via searching using node-filters “proteobacteria”, “firmicutes” and “actinobacteria”. LCC and PETase enzymes had UniProt entries under G9BY57 and A0A0K8P6T7 respectively, and these ascension codes were input into the Cytoscape search function to select and highlight their respective nodes in the SSN. As SIBER 1-4 are novel proteins with no UniProt entries, proxy proteins with high levels of similarities was used to visualize their relative positions within the SSN. The individual amino acid sequences of each SIBER protein was put through BLAST against UniProt database to select a proxy entry using two criteria: having the highest % similarity and was documented to perform similar enzymatic functions. The Alpha/Beta Hydrolase from Microbispora hainanensis (actinobacteria) was selected as the proxy for SIBER 1 at 99.22% similarity (UniProt A0A544Z467). SIBER 2-4 proxies had 93.87%, 95.91% and 90% similarities respectively (UniProt A0A1C4ULM8, A0A1N6VAB5, A0A1C5GKF5).

To expand sequence space for plastic breakdown and modifying enzymes, we have mined 4 unique putative cutinases from Actinobacteria. Their sequence similarity distance from known PETase and cutinases is ~60%.

In order to express and characterize these enzymes, we have to engineer various solubility and his-tag arrangements to optimize protein solubility, folding and thus activity. Out of these, one particular construct, SIBER 171 was able to breakdown high crystallinity PET at 25 °C and 45 °C.

### Example 1: Identification, Characterisation and Expression of Actinobacterial Hydrolases

### Genomic Mining and in Silico Analysis of Distant Cutinase Homologs

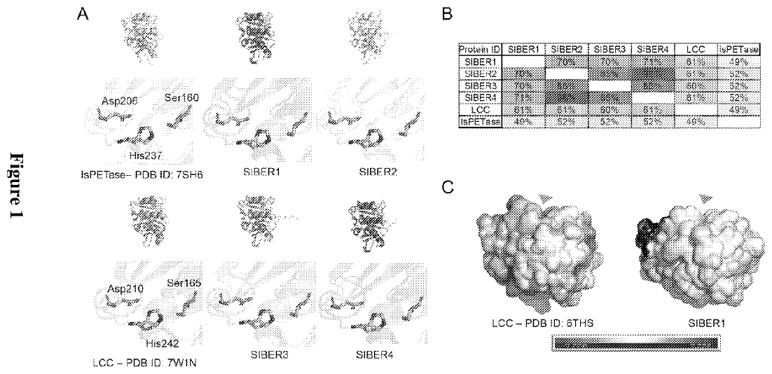

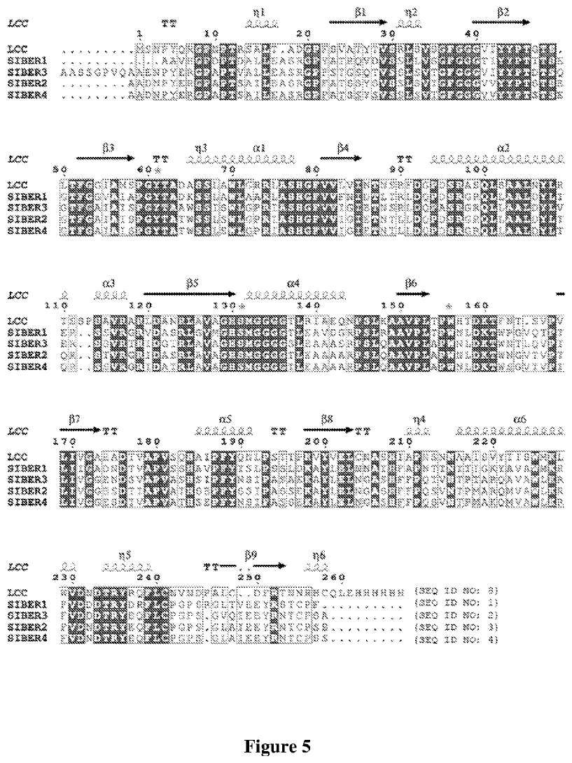

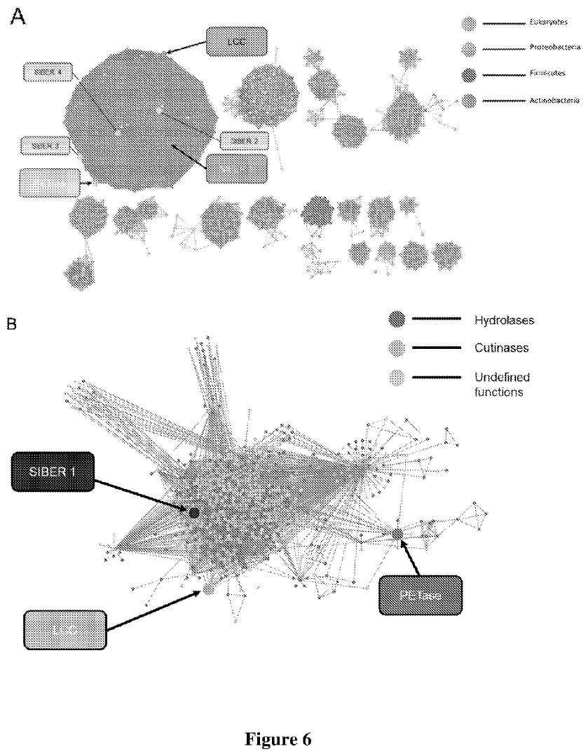

With reference to the reported LCC, potential hydrolases were obtained from the genera Microbispora, Nonomuraea, and Micromonospora through mining actinobacterial genomic sequences in public databases such as GenBank database, as well as the Singapore-based Natural Organism Library (NOL). Within these hits, four representative sequences were initially selected from NOL. Sequence alignment analysis between four of the hydrolases indicate sequence similarity to leaf-branch compost cutinase (LCC) (60% homology, FIG. 1), including the presence of the three active site triad residues for PET breakdown (Asp, Ser and His, FIG. 1A). Cysteine residues for disulphide bridge formation were also observed (FIG. 5). Search within the ESTHER database also showed high similarity hits of these enzymes as part of the polyesterase-lipase-cutinase family—similar to many previously reported PET hydrolases. (FIG. 6 SSN). Absence of an extended loop and extra disulphide bond near the active site also classify these enzymes to be Type I (LCC) rather than Type II (IsPETase). Structural alignment based on in silico AlphaFold models also proposed that their general structure is closely aligned to that of the LCC, including positions of the active site residues. Although the core active sites and general structures are conserved, electrostatic prediction of the surface indicated sequence divergence are mainly surface residues. This observation is similar to that from an extensive study on PET breakdown enzymes.

### Protein Expression Optimisation

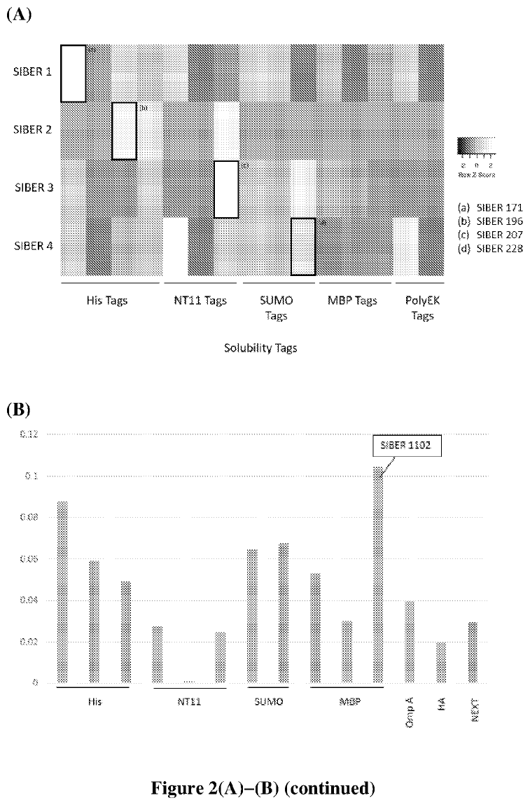

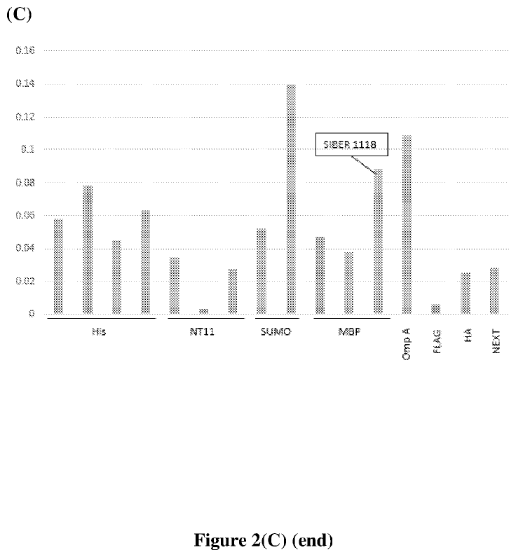

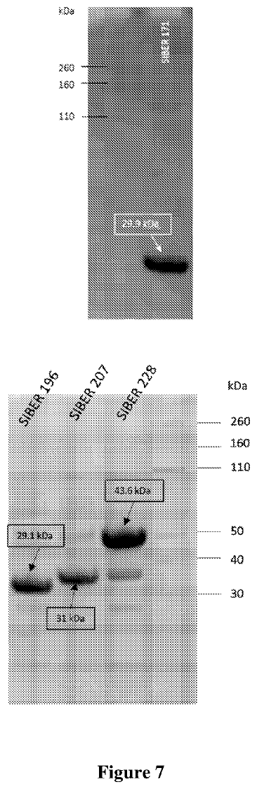

To investigate if the shortlisted sequences encode functional enzymes, there is a need to identify a suitable expression system. For heterologous expression of these enzymes, sequence optimization was first done for the protein expression constructs of SIBER 1-4. These were screened with various fusion solubility tags (NT11, SUMO, MBP, his) for optimal expression (FIG. 2). Solubility aids included SUMO and a 11 amino acid tag, NT11 were investigated. Our optimization data showed varied expression levels across enzymes and tag combination (FIG. 2). Overall, SIBER 1 constructs had significantly higher expression levels. The 5 best expressing constructs which comprises of at least 1 construct from each of the unique sequences (SIBER 171, SIBER 196, SIBER 207, SIBER 228), were further scaled up and the resulting proteins were purified for characterisation (FIG. 7). This scale up resulted in the yield of these 5 proteins ranging from 3-8 mg/L.

### Characterisation of SIBER 171, 196, 207, 228, 1118, 1102, 1969, 1976

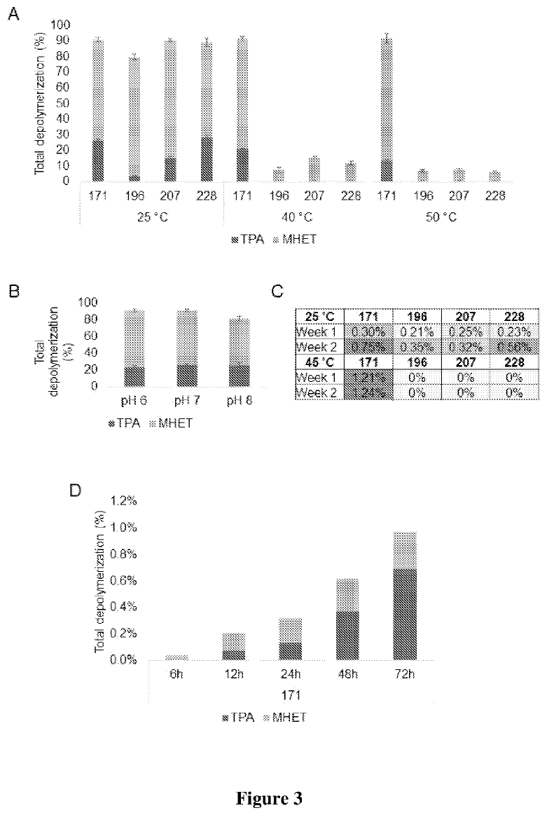

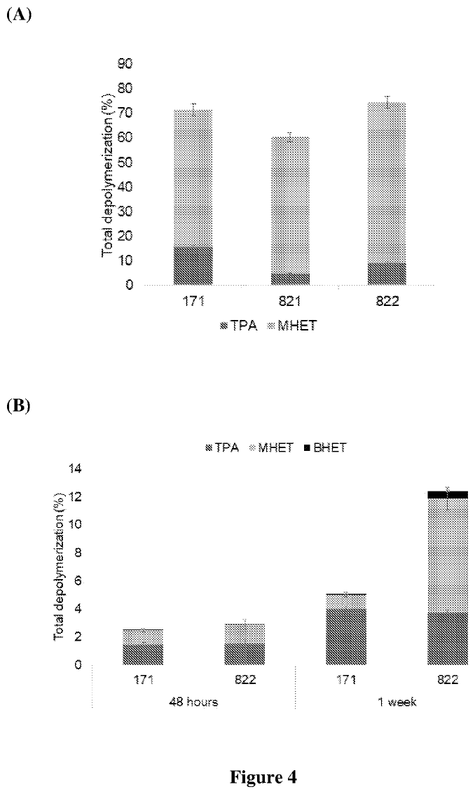

In order to assess the functional activity of the enzymes, the purified enzymes (SIBER 171, 196, 207, 228, 1118, 1102, 1969 or 1976) were subjected to various assays using different substrates (e.g. BHET, hcPET, lcPET). Under ambient temperature (25 °C) at pH 7, all the enzymes were able to hydrolyze BHET to various extents (FIG. 3). The best mutant was SIBER 171 which resulted in ~95% depolymerisation by the end of 24 h. SIBER 171 was also functional up to 50 °C, whereas SIBER 196, SIBER 207 and SIBER 228 were only functional at 25 °C. Further characterization of SIBER 171 also showed that their activities are maintained across pH 6-8.

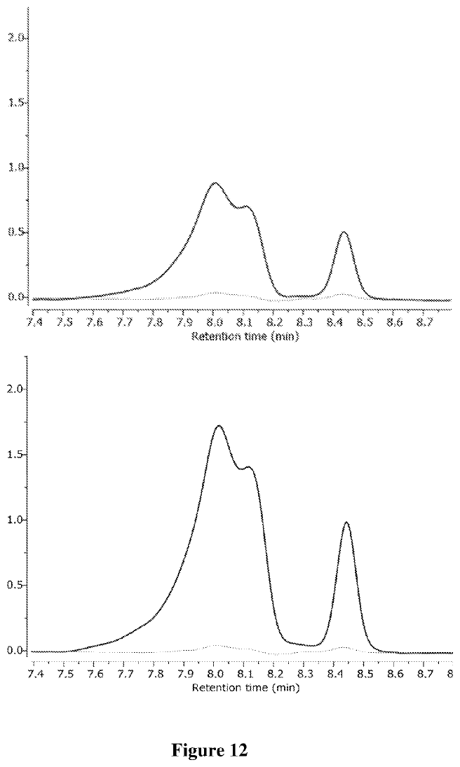

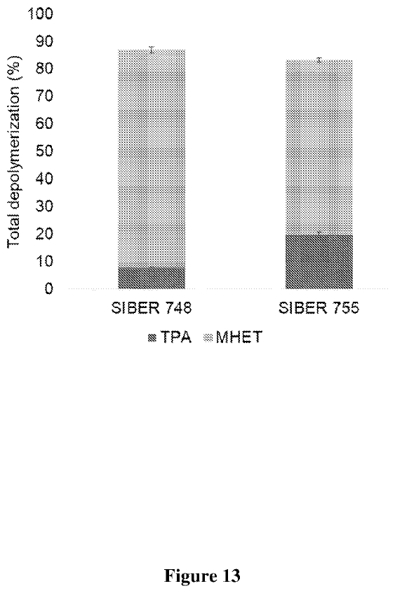

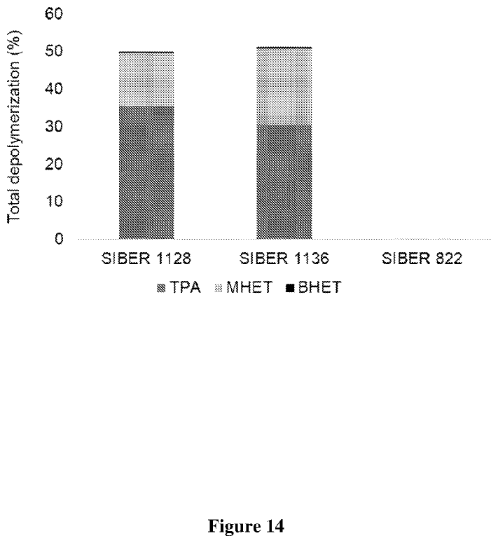

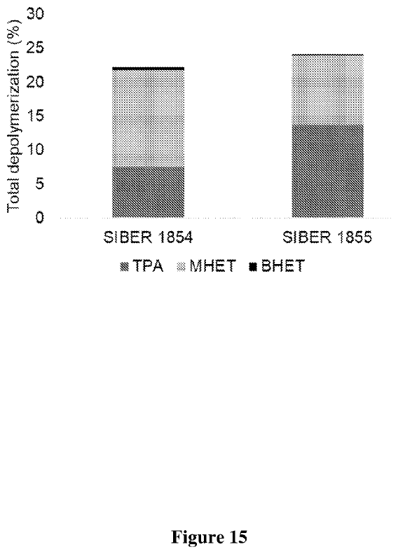

In addition to these initial 4 sequences, a further three sequences within the same genus were also examined. SIBER 1102 (Nonomuraea) and SIBER 1118 (Microbispora) demonstrated depolymerisation of low crystallinity PET (FIG. 12) at 70 °C for 6 hours. Another wild type sequence (Microbiospora) was also used to demonstrate depolymerisation of BHET at 25 °C, pH 8, 24 hours; where we observed 47% (SIBER 1969, 46.1% MHET and 1.1% TPA) and 29.1% (SIBER 1976, 28.6% MHET and 0.5% TPA) depolymerisation.

Applying the reported optimised conditions for leaf-branch compost cutinase (LCC) (70 °C, 48 h) for PET depolymerisation to these enzymes did not show any PET breakdown, suggesting that the native enzymes are not tolerable to such high temperatures. Re-assaying these enzymes at lower temperatures (25 °C or 45 °C) over longer time (up to 3 weeks), we found that all five enzymes were able to assimilate high crystallinity PET (hcPET), though at a very slow rate (FIG. 3C). The best performing enzymes herein are SIBER 171. Conversions mostly doubled in the 2nd week when compared to week 1, indicating that the enzyme was still active. None of the variants are sufficiently thermostable to effectively break down high crystallinity (>35%) PET at 45 °C, though small amount of PET conversion was observed at 45 °C after 1 week for the enzymes SIBER 171, there was no further conversion after 1 week, suggesting that SIBER 171 might have been inactivated.

### Example 2: Engineering of SIBER 1

To determine if similar optimisations made to LCC can be applied to improve SIBER 1, additional mutations were introduced, including the insertion of a disulphide bond for increased thermal stability (D238C/S283C, as numbered in the Tournier et al. paper (Tournier et al., Nature 580, pg. 216-219 (2020)), which correspond to positions N202/T247 of SEQ ID NO: 1), the F243I mutation (as numbered in the Tournier et al. paper, which corresponds to position F207 of SEQ ID NO: 1) to restore activity and the N246M mutation (as numbered in the Tournier et al. paper, which corresponds to position N210 of SEQ ID NO: 1) for further optimisation. Similar mutations were applied to SIBER 171 (His-tag-containing SIBER 1) giving SIBER 821 and 822 (Table 3). These were then characterised for BHET (50 °C) and PET (45 °C, 65 °C) breakdown.

During BHET hydrolysis (50 °C, FIG. 1), detrimental effects were observed for LCC mutations which were however restored with the N246M mutation (FIG. 1). Among these mutants, SIBER 822 had the best activity. Using low crystallinity PET (lcPET) substrates at 45 °C, there appeared to be significant improvement of SIBER 822 over SIBER 171 over 1 week (FIG. 1). At 65 °C, no PET depolymerisation was observed for both enzymes. These observations suggest that, despite not being enough to increase its working temperature, the addition of a disulphide bridge to SIBER 1 resulted in significantly better activity at 45 °C over 1 week when compared to native enzymes.

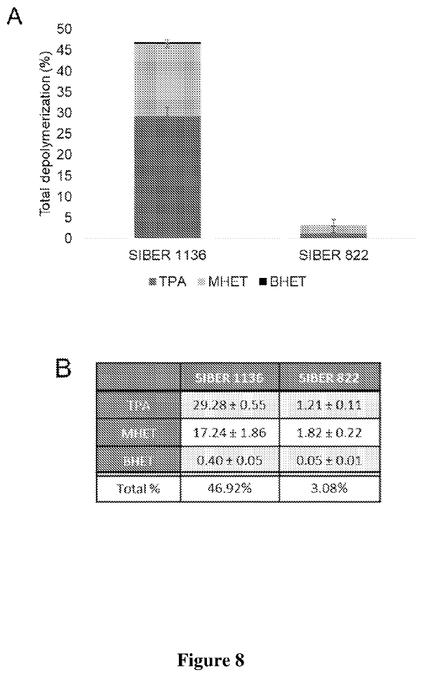

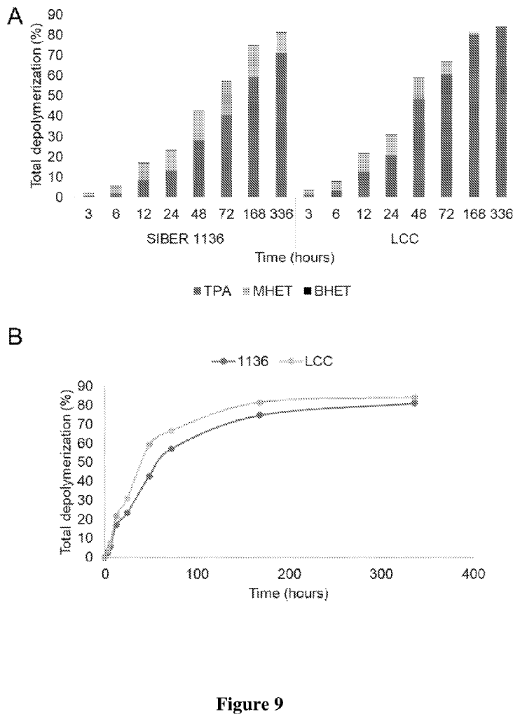

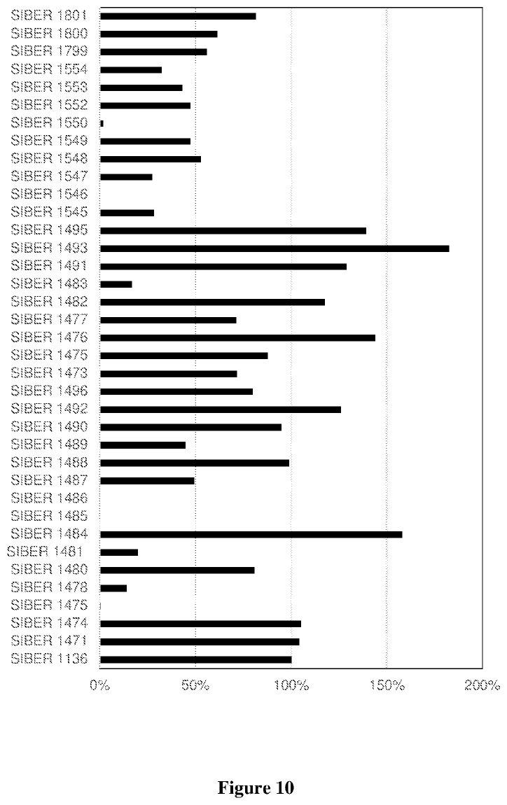

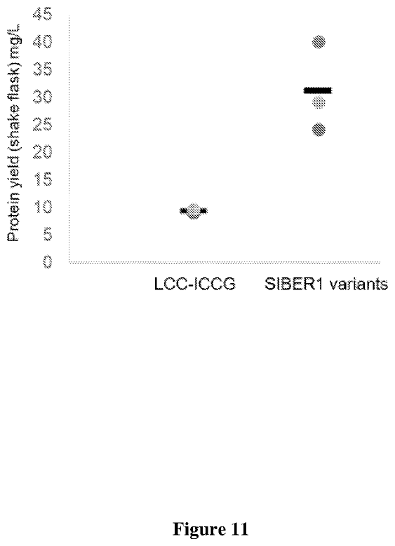

Further variants were engineered by introducing additional substitutions in SIBER 822, taking reference from the amino acid sequence of LCC, as shown in Table 3. SIBER 1136 showed improved activity over SIBER 822 for depolymerisation of lcPET, achieving a total depolymerisation that is more than 15-fold higher than SIBER 822 (FIG. 8). SIBER 1136 also exhibited comparable depolymerisation activity to LCC when depolymerising lcPET over 2 weeks (45 °C, pH 8) (FIG. 9).

This study aimed to enhance the understanding of PET-degrading enzymes by identifying and describing new ones that can aid in PET waste biodegradation. Although some PET-degrading enzymes are known, their rarity restricts their potential use. Even with the addition of 34 more recently characterized PET degrading enzymes from the actinobacteria family, this still adds up to only a few dozen of verified PET-active enzymes. Here, new hydrolases were discovered from a new genus space (Microbispora, Nonomuraea, and Micromonospora) with varying specificities towards substrates BHET, MHET, and even PET. Within representative sequences, a new thermostable PETase has been characterised.

To date, there are over 2000 known PET homologs and several enzyme engineering studies have proposed targeted mutations to improve thermostability, activity, and accessibility to PET. Among the seven enzymes, there were notable differences in solubility and expression yields, leading to the investigation of the correlation between protein folding and protein activity, where the thermodynamics of the sequences may play a role in proper and accurate folding.

TABLE 1
Depolymerisation (%) of low crystallinity PET
(Goodfellow amorphous film) by
IsPETase at 45° C. over 48 hours and over 1 week.

| IsPETase | 48 h | 1 week |
|---|---|---|
| TPA | 0.00 ± 0.00 | 0.00 ± 0.00 |
| MHET | 0.09 ± 0.01 | 0.08 ± 0.01 |
| BHET | 0.00 ± 0.00 | 0.00 ± 0.00 |
| Total depolymerised | 0.09% | 0.08% |

TABLE 2
Depolymerisation of low crystallinity PET by SIBER 1136 and LCC-ICCG at 45 °C and
pH 8 over 2 weeks, as reflected in the production of degradation products TPA, MHET and BHET.

| | SIBER 1136 | | | | | | | |
|---|---|---|---|---|---|---|---|---|
| Hours | 3 | 6 | 12 | 24 | 48 | 72 | 168 | 336 |
| TPA | 0.7% | 1.9% | 8.4% | 13.3% | 28.1% | 40.7% | 59.3% | 71.0% |
| MHET | 1.6% | 3.6% | 8.4% | 9.8% | 14.3% | 16.1% | 15.2% | 9.9% |
| BHET | 0.0% | 0.1% | 0.2% | 0.2% | 0.3% | 0.3% | 0.3% | 0.2% |
| Total | 2.3% | 5.6% | 17.1% | 23.3% | 42.7% | 57.1% | 74.7% | 81.0% |
| | LCC | | | | | | | |
| Hours | 3 | 6 | 12 | 24 | 48 | 72 | 168 | 336 |
| TPA | 1.2% | 3.2% | 12.3% | 20.7% | 48.7% | 60.6% | 80.3% | 84.0% |
| MIHET | 2.1% | 4.4% | 9.1% | 9.9% | 10.2% | 5.9% | 1.2% | 0.2% |
| BHET | 0.1% | 0.1% | 0.3% | 0.3% | 0.3% | 0.1% | 0.0% | 0.0% |
| Total | 3.3% | 7.8% | 21.7% | 30.9% | 59.2% | 66.6% | 81.5% | 84.2% |

TABLE 3
Amino acid sequences of wild-type and variant enzymes.

| Enzyme | Modification | Amino acid sequence |
|---|---|---|
| SIBER 1 | Wild-type | AAVHGPDPTDALLEASRGPYATRQVDVSSLLVSGFGGGT |
| | (Microbispora | IYYPTTTSEGTFGGVAIAPGYTADKSSLAWLAARLASHGF |
| | hainanensis str. | VVFNIDTLTRLDQPDSRGRQLLAALDYLTERSSVRGRVD |
| | A3051) | ASRLGVMGHSMGGGGTLEAVDGRPSLRAAVPLTPWNLD |
| | | KTWPGVQTPTLIIGADNDTVAPVSSHAVPFYTSLPSSLDK |
| | | AYLELNNATHFAPNTTNTTIGKYAVAWMKRFVDDDTRY |
| | | DRFLCPGPSRGLTVEEYRSTCPF |
| | | (SEQ ID NO: 1) |
| SIBER 2 | Wild-type | AADNPYERGPAPTSAILEASRGPFATSSYSVSSLSVTGFGG |
| | (Nonomuraea sp. | GVVYYPTSTSEGTFGAIAISPGYTATWSSLSWLGPRIASH |
| | TT08I-71 str. | GFVVIGIETNTLLDQPDSRGRQLLAALDYLTQRSTVRGRI |
| | T589) | DASRLAVAGHSMGGGGTLEAAAARPSLQAAVPLAPWNL |
| | | DKTWNGVTVPTLIVGGESDTIAPVATHSEPFYSSIPASSEK |
| | | AYLELNGASHFFPQSVNTPMARQMVAWLKRFVDNDTRY |
| | | EQFLCPGPSGLAIEEYRNTCPSS |
| | | (SEQ ID NO: 2) |
| SIBER 3 | Wild-type | AASSGPVQAAENPYERGPAPTVALLEASRGPFSTGSQTVS |
| | (Micromonospora | SLSVTGFGGGVIYYPTDTSQGTFGAIAISPGYTAAWSSISW |
| | musae str. T593) | LGPRIASHGFVVIGIETNSRLDQPASRGRQLLAALDYLVE |
| | | RSSVRTRIDGTRLAVAGHSMGGGGSLEAAASRPSLQAAV |
| | | PLAPWNLDKTWNTLQVPTLIVGGENDSVAPVASHSIPFY |
| | | NSIPASAEKAYLELNGASHFFPQTVNTPTARQAVAWLKR |
| | | FVDDDTRYEQFLCPGPSGVQIEEYRNTCPSA |
| | | (SEQ ID NO: 3) |
| SIBER 4 | Wild-type | AADNPYERGPAPTSAILEASRGPFATSSYSVSSLSVTGFGG |
| | (Micromonospora | GVVYYPTSTSEGTFGAIAISPGYTATWSSLSWLGPRIASH |
| | chersina str. | GFVVIGIETNTLLDQPDSRGRQLLAALDYLTQRSSVRGRI |
| | A2592) | DATRLAVAGHSMGGGGTLEAAAARPSLQAAVPLAPWNL |
| | | DKTWSGVTVPTLIVGGESDTVAPVATHSEPFYNSIPASSE |
| | | KAYLELNGASHFFPQSVNTPMAKQMVAWLKRFVDNDTR |
| | | YEQFLCPGPSGLAIEEYRNTCPSS |
| | | (SEQ ID NO: 4) |
| SIBER | Wild-type | AAVHGPDPTDALLESSRGPYATAQTDVSSLSASGFGGGTI |
| 1118WT | (Microbispora | YYPTTTSEGTFGGVAIAPGYTADKSSLAWLAARLASHGF |
| | camponoti) | VVFNIDTLTRLDQPDSRGRQLLAALDYLTQRSSVRGRVD |
| | | ATRLGVMGHSMGGGGTLEAVDDRPSLRAAVPLTPWNLD |
| | | KTWSGVRTPTLIIGAEADTVAPVVSHAVPFYTSLSSSLDK |
| | | AYLELNGATHFAPNTTNTTIGKYAVAWMKRFVDDDTRY |
| | | DQFLCPGPSRSLTVEEYRSTCPF |
| | | (SEQ ID NO: 5) |
| SIBER | Wild-type | ASASPYQRGPDPTEAVLEASRGPFSVSDTTVSSLVVTGFG |
| 1102WT | (Nonomuraea | GGTIYYPTSTAEGTFGGVAIAPGYTASRSSMAWLAERIAS |
| | soli) | HGFVVFNIDTITRSDQPASRGRQLLAALDYLTERSTVRSR |
| | | LDASRLGVMGHSMGGGGTLEASASRPSLQAAVPLTPWN |
| | | LDKSWSEVQVPTMIIGAENDSIASVTSHSEPFYNSLPSSLD |
| | | KAYLELDGASHFAPNTTNPTIGKFSVAWLKRFIDDDLRYE |
| | | QFLCPPPVGDRDISEYRNTCPHS |
| | | (SEQ ID NO: 6) |
| SIBER | Wild type | AAHAAVHGPDPTDALLEASRGPYATAQVDVSSLLVSGFG |
| 1969/ | (Microbispora) | GGTIYYPATTSEGTFGGVAIAPGYTADKSSLAWLAARLA |
| SIBER | | SHGFVVFNIDTLTRLDQPDSRGRQLLAALDYLTQRSSVRG |
| 1976 | | RVDATRLGVMGHSMGGGGTLEAVDDRPSLRAAVPLTPW |
| | | NLDKTWSGVRTPTLIIGADNDTIAPVASHAVPFYTSLPSSL |
| | | DKAYLELNNATHFAPNTTNTTIGKYAVAWMKRFIDDDT |
| | | RYDGFLCPGPDRGLTVEEYRSTCPF |
| | | (SEQ ID NO: 7) |
| Leaf | Engineered | MSNPYQRGPNPTRSALTADGPFSVATYTVSRLSVSGFGG |
| branch | (Tournier et al., | GVIYYPTGTSLTFGGIAMSPGYTADASSLAWLGRRLASH |
| compost | Nature 580) | GFVVLVINTNSRFDGPDSRASQLSAALNYLRTSSPSAVRA |
| cutinase | | RLDANRLAVAGHAMGGGGTLRIAEQNPSLKAAVPLTPW |
| (LCC) | | HTDKTFNTSVPVLIVGAEADTVAPVSQHAIPFYQNLPSTT |
| | | PKVYVELCNASHIAPNSNNAAISVYTISWMKLWVDNDTR |
| | | YRQFLCNVNDPALCDFRTNNRHCQLEHHHHHH |
| | | (SEQ ID NO: 8) |
| SIBER | His tag | MGSSHHHHHHAGAAAVHGPDPTDALLEASRGPYATRQV |
| 171 | | DVSSLLVSGFGGGTIYYPTTTSEGTFGGVAIAPGYTADKS |
| | | SLAWLAARLASHGFVVFNIDTLTRLDQPDSRGRQLLAAL |
| | | DYLTERSSVRGRVDASRLGVMGHSMGGGGTLEAVDGRP |
| | | SLRAAVPLTPWNLDKTWPGVQTPTLIIGADNDTVAPVSS |
| | | HAVPFYTSLPSSLDKAYLELNNATHFAPNTTNTTIGKYAV |
| | | AWMKRFVDDDTRYDRFLCPGPSRGLTVEEYRSTCPFAGA |
| | | HHHHHH |
| | | (SEQ ID NO: 9) |
| SIBER | His tag | MGSSHHHHHHAGAAADNPYERGPAPTSAILEASRGPFAT |
| 196 | | SSYSVSSLSVTGFGGGVVYYPTSTSEGTFGAIAISPGYTAT |
| | | WSSLSWLGPRIASHGFVVIGIETNTLLDQPDSRGRQLLAA |
| | | LDYLTQRSTVRGRIDASRLAVAGHSMGGGGTLEAAAAR |
| | | PSLQAAVPLAPWNLDKTWNGVTVPTLIVGGESDTIAPVA |
| | | THSEPFYSSIPASSEKAYLELNGASHFFPQSVNTPMARQM |
| | | VAWLKRFVDNDTRYEQFLCPGPSGLAIEEYRNTCPSSAG |
| | | AHHHHHH |
| | | (SEQ ID NO: 10) |
| SIBER | NT11 tag | MVSEPHDYNYEKAGAAASSGPVQAAENPYERGPAPTVA |
| 207 | | LLEASRGPFSTGSQTVSSLSVTGFGGGVIYYPTDTSQGTFG |
| | | AIAISPGYTAAWSSISWLGPRIASHGFVVIGIETNSRLDQP |
| | | ASRGRQLLAALDYLVERSSVRTRIDGTRLAVAGHSMGGG |
| | | GSLEAAASRPSLQAAVPLAPWNLDKTWNTLQVPTLIVGG |
| | | ENDSVAPVASHSIPFYNSIPASAEKAYLELNGASHFFPQTV |
| | | NTPTARQAVAWLKRFVDDDTRYEQFLCPGPSGVQIEEYR |
| | | NTCPSAAGAHHHHHH |
| | | (SEQ ID NO: 11) |
| SIBER | SUMO tag | MGGGSSEHEHEHSSGLVPRGSHMASMSDSEVNQEAKPE |
| 228 | | VKPEVKPETHINLKVSDGSSEIFFKIKKTTPLRRLMEAFAK |
| | | RQGKEMDSLRFLYDGIRIQADQTPEDLDMEDNDIIEAHRE |
| | | QIGGSAGAETASSEGLAGAAADNPYERGPAPTSAILEASR |
| | | GPFATSSYSVSSLSVTGFGGGVVYYPTSTSEGTFGAIAISP |
| | | GYTATWSSLSWLGPRIASHGFVVIGIETNTLLDQPDSRGR |
| | | QLLAALDYLTQRSSVRGRIDATRLAVAGHSMGGGGTLE |
| | | AAAARPSLQAAVPLAPWNLDKTWSGVTVPTLIVGGESDT |
| | | VAPVATHSEPFYNSIPASSEKAYLELNGASHFFPQSVNTP |
| | | MAKQMVAWLKRFVDNDTRYEQFLCPGPSGLAIEEYRNT |
| | | CPSSAGAHHHHHH |
| | | (SEQ ID NO: 12) |
| SIBER | His tag, N202C, | MGSSHHHHHHAGAAAVHGPDPTDALLEASRGPYATRQV |
| 821 | F207I, T247C | DVSSLLVSGFGGGTIYYPTTTSEGTFGGVAIAPGYTADKS |
| | | SLAWLAARLASHGFVVFNIDTLTRLDQPDSRGRQLLAAL |
| | | DYLTERSSVRGRVDASRLGVMGHSMGGGGTLEAVDGRP |
| | | SLRAAVPLTPWNLDKTWPGVQTPTLIIGADNDTVAPVSS |
| | | HAVPFYTSLPSSLDKAYLELCNATHIAPNTTNTTIGKYAV |
| | | AWMKRFVDDDTRYDRFLCPGPSRGLCVEEYRSTCPFAG |
| | | AHHHHHH |
| | | (SEQ ID NO: 13) |
| SIBER | His tag, N202C, | MGSSHHHHHHAGAAAVHGPDPTDALLEASRGPYATRQV |
| 822 | F207I, N210M, | DVSSLLVSGFGGGTIYYPTTTSEGTFGGVAIAPGYTADKS |
| | T247C | SLAWLAARLASHGFVVFNIDTLTRLDQPDSRGRQLLAAL |
| | | DYLTERSSVRGRVDASRLGVMGHSMGGGGTLEAVDGRP |
| | | SLRAAVPLTPWNLDKTWPGVQTPTLIIGADNDTVAPVSS |
| | | HAVPFYTSLPSSLDKAYLELCNATHIAPMTTNTTIGKYAV |
| | | AWMKRFVDDDTRYDRFLCPGPSRGLCVEEYRSTCPFAG |
| | | AHHHHHH |
| | | (SEQ ID NO: 14) |
| SIBER | MBP tag, N202C, | MKYYKHEHEHEHEHEHEHEGASGMKIEEGKVVIWHAM |
| 1118 | F207I, N210M, | QPNELEVFQSLAEEYMALCPEVEIVFEQKPNLEDALKAAI |
| | T247C | PTGQGPDLFIWAHDWIGKFAEAGLLEPIDEYVTEDLLNEF |
| | | APMAQDAMQYKGHYYALPFAAETVAIIYNKEMVSEPPK |
| | | TFDEMKAIMEKYYDPANEKYGIAWPINAYFISAIAQAFGG |
| | | YYFDDKTEQPGLDKPETIEGFKFFFTEIWPYMAPTGDYNT |
| | | QQSIFLEGRAPMMVNGPWSINDVKKAGINFGVVPLPPIIK |
| | | DGKEYWPRPYGGVKLIYFAAGIKNKDAAWKFAKWLTTS |
| | | EESIKTLALELGYIPVLTKVLDDPEIKNDPVIYGFGQAVQH |
| | | AYLMPKSPKMSAVWGGVDGAINEILQDPQNADIEGILKK |
| | | YQQEILNNMQGEENLYFQSIAGAAAVHGPDPTDALLESS |
| | | RGPYATAQTDVSSLSASGFGGGTIYYPTTTSEGTFGGVAI |
| | | APGYTADKSSLAWLAARLASHGFVVFNIDTLTRLDQPDS |
| | | RGRQLLAALDYLTQRSSVRGRVDATRLGVMGHSMGGG |
| | | GTLEAVDDRPSLRAAVPLTPWNLDKTWSGVRTPTLIIGAE |
| | | ADTVAPVVSHAVPFYTSLSSSLDKAYLELCGATHIAPMTT |
| | | NTTIGKYAVAWMKRFVDDDTRYDQFLCPGPSRSLCVEE |
| | | YRSTCPFAGAHHHHHH |
| | | (SEQ ID NO: 15) |
| SIBER | MBP tag, F24Y, | MKYYKHEHEHEHEHEHEHEGASGMKIEEGKVVIWHAM |
| 1102 | V35L, T37S, | QPNELEVFQSLAEEYMALCPEVEIVFEQKPNLEDALKAAI |
| | S70T, A79T, | PTGQGPDLFIWAHDWIGKFAEAGLLEPIDEYVTEDLLNEF |
| | T116S, A143S, | APMAQDAMQYKGHYYALPFAAETVAIIYNKEMVSEPPK |
| | S185T D206C, | TFDEMKAIMEKYYDPANEKYGIAWPINAYFISAIAQAFGG |
| | F211I, N214M, | YYFDDKTEQPGLDKPETIEGFKFFFTEIWPYMAPTGDYNT |
| | D249G, R250L | QQSIFLEGRAPMMVNGPWSINDVKKAGINFGVVPLPPIIK |
| | D251C | DGKEYWPRPYGGVKLIYFAAGIKNKDAAWKFAKWLTTS |
| | | EESIKTLALELGYIPVLTKVLDDPEIKNDPVIYGFGQAVQH |
| | | AYLMPKSPKMSAVWGGVDGAINEILQDPQNADIEGILKK |
| | | YQQEILNNMQGEENLYFQSIAGAASASPYQRGPDPTEAV |
| | | LEASRGPYSVSDTTVSSLLVSGFGGGTIYYPTSTAEGTFG |
| | | GVAIAPGYTASRSTMAWLAERITSHGFVVFNIDTITRSDQ |
| | | PASRGRQLLAALDYLTERSSVRSRLDASRLGVMGHSMG |
| | | GGGTLEASSSRPSLQAAVPLTPWNLDKSWSEVQVPTMIIG |
| | | AENDSIASVTTHSEPFYNSLPSSLDKAYLELCGASHIAPMT |
| | | TNPTIGKFSVAWLKRFIDDDLRYEQFLCPPPVGGLCISEYR |
| | | NTCPHSAGAHHHHHH |
| | | (SEQ ID NO: 16) |
| SIBER | N202C, F207I, | AAVHGPDPTDALLEASRGPYATRQVDVSSLLVSGFGGGT |
| 822* | N210M, T247C | IYYPTTTSEGTFGGVAIAPGYTADKSSLAWLAARLASHGF |
| | | VVFNIDTLTRLDQPDSRGRQLLAALDYLTERSSVRGRVD |
| | | ASRLGVMGHSMGGGGTLEAVDGRPSLRAAVPLTPWNLD |
| | | KTWPGVQTPTLIIGADNDTVAPVSSHAVPFYTSLPSSLDK |
| | | AYLELCNATHIAPMTTNTTIGKYAVAWMKRFVDDDTRY |
| | | DRFLCPGPSRGLCVEEYRSTCPF |
| | | (SEQ ID NO: 17) |
| SIBER | N202C, F207I, | AAVHGPDPTDALLESSRGPYATAQTDVSSLSASGFGGGTI |
| 1118* | N210M, T247C | YYPTTTSEGTFGGVAIAPGYTADKSSLAWLAARLASHGF |
| | | VVFNIDTLTRLDQPDSRGRQLLAALDYLTQRSSVRGRVD |
| | | ATRLGVMGHSMGGGGTLEAVDDRPSLRAAVPLTPWNLD |
| | | KTWSGVRTPTLIIGAEADTVAPVVSHAVPFYTSLSSSLDK |
| | | AYLELCGATHIAPMTTNTTIGKYAVAWMKRFVDDDTRY |
| | | DQFLCPGPSRSLCVEEYRSTCPF |
| | | (SEQ ID NO: 18) |
| SIBER | F24Y, V35L, | ASASPYQRGPDPTEAVLEASRGPYSVSDTTVSSLLVSGFG |
| 1102* | T37S, S70T, | GGTIYYPTSTAEGTFGGVAIAPGYTASRSTMAWLAERITS |
| | A79T, T116S, | HGFVVFNIDTITRSDQPASRGRQLLAALDYLTERSSVRSR |
| | A143S, S185T | LDASRLGVMGHSMGGGGTLEASSSRPSLQAAVPLTPWN |
| | D206C, F211I, | LDKSWSEVQVPTMIIGAENDSIASVTTHSEPFYNSLPSSLD |
| | N214M, D249G, | KAYLELCGASHIAPMTTNPTIGKFSVAWLKRFIDDDLRYE |
| | R250L D251C | QFLCPPPVGGLCISEYRNTCPHS |
| | | (SEQ ID NO: 19) |
| SIBER | K64A, L90F, | AAVHGPDPTDALLEASRGPYATRQVDVSSLLVSGFGGGT |
| 1136 | Q92Y, E136R, | IYYPTTTSEGTFGGVAIAPGYTADASSLAWLAARLASHGF |
| | N154H, | VVFNIDTLTRFDYPDSRGRQLLAALDYLTERSSVRGRVD |
| | N202C, F207I, | ASRLGVMGHSMGGGGTLRAVDGRPSLRAAVPLTPWHLD |
| | N210M, T247C | KTWPGVQTPTLIIGADNDTVAPVSSHAVPFYTSLPSSLDK |
| | | AYLELCNATHIAPMTTNTTIGKYAVAWMKRFVDDDTRY |
| | | DRFLCPGPSRGLCVEEYRSTCPF |
| | | (SEQ ID NO: 20) |
| SIBER | H4K, K64A, | AAVKGPDPTDALLEASRGPYATRQVDVSSLLVSGFGGGT |
| 1471 | L90F, Q92Y, | IYYPTTTSEGTFGGVAIAPGYTADASSLAWLAARLASHGF |
| | E136R, N154H, | VVFNIDTLTRFDYPDSRGRQLLAALDYLTERSSVRGRVD |
| | N202C, F207I, | ASRLGVMGHSMGGGGTLRAVDGRPSLRAAVPLTPWHLD |
| | N210M, T247C | KTWPGVQTPTLIIGADNDTVAPVSSHAVPFYTSLPSSLDK |
| | | AYLELCNATHIAPMTTNTTIGKYAVAWMKRFVDDDTRY |
| | | DRFLCPGPSRGLCVEEYRSTCPF |
| | | (SEQ ID NO: 21) |
| SIBER | L30A, K64A, | AAVHGPDPTDALLEASRGPYATRQVDVSSALVSGFGGGT |
| 1473 | L90F, Q92Y, | IYYPTTTSEGTFGGVAIAPGYTADASSLAWLAARLASHGF |
| | E136R, N154H, | VVFNIDTLTRFDYPDSRGRQLLAALDYLTERSSVRGRVD |
| | N202C, F207I, | ASRLGVMGHSMGGGGTLRAVDGRPSLRAAVPLTPWHLD |
| | N210M, T247C | KTWPGVQTPTLIIGADNDTVAPVSSHAVPFYTSLPSSLDK |
| | | AYLELCNATHIAPMTTNTTIGKYAVAWMKRFVDDDTRY |
| | | DRFLCPGPSRGLCVEEYRSTCPF |
| | | (SEQ ID NO: 22) |
| SIBER | L31A, K64A, | AAVHGPDPTDALLEASRGPYATRQVDVSSLAVSGFGGGT |
| 1474 | L90F, Q92Y, | IYYPTTTSEGTFGGVAIAPGYTADASSLAWLAARLASHGF |
| | E136R, N154H, | VVFNIDTLTRFDYPDSRGRQLLAALDYLTERSSVRGRVD |
| | N202C, F207I, | ASRLGVMGHSMGGGGTLRAVDGRPSLRAAVPLTPWHLD |
| | N210M, T247C | KTWPGVQTPTLIIGADNDTVAPVSSHAVPFYTSLPSSLDK |
| | | AYLELCNATHIAPMTTNTTIGKYAVAWMKRFVDDDTRY |
| | | DRFLCPGPSRGLCVEEYRSTCPF |
| | | (SEQ ID NO: 23) |
| SIBER | T61G, K64A, | AAVHGPDPTDALLEASRGPYATRQVDVSSLLVSGFGGGT |
| 1477 | L90F, Q92Y, | IYYPTTTSEGTFGGVAIAPGYGADASSLAWLAARLASHGF |
| | E136R, N154H, | VVFNIDTLTRFDYPDSRGRQLLAALDYLTERSSVRGRVD |
| | N202C, F207I, | ASRLGVMGHSMGGGGTLRAVDGRPSLRAAVPLTPWHLD |
| | N210M, T247C | KTWPGVQTPTLIIGADNDTVAPVSSHAVPFYTSLPSSLDK |
| | | AYLELCNATHIAPMTTNTTIGKYAVAWMKRFVDDDTRY |
| | | DRFLCPGPSRGLCVEEYRSTCPF |
| | | (SEQ ID NO: 24) |
| SIBER | K64A, L90F, | AAVHGPDPTDALLEASRGPYATRQVDVSSLLVSGFGGGT |
| 1482 | Q92G, E136R, | IYYPTTTSEGTFGGVAIAPGYTADASSLAWLAARLASHGF |
| | N154H, | VVFNIDTLTRFDGPDSRGRQLLAALDYLTERSSVRGRVD |
| | N202C, F207I, | ASRLGVMGHSMGGGGTLRAVDGRPSLRAAVPLTPWHLD |
| | N210M, T247C | KTWPGVQTPTLIIGADNDTVAPVSSHAVPFYTSLPSSLDK |
| | | AYLELCNATHIAPMTTNTTIGKYAVAWMKRFVDDDTRY |
| | | DRFLCPGPSRGLCVEEYRSTCPF |
| | | (SEQ ID NO: 25) |
| SIBER | K64A, L90F, | AAVHGPDPTDALLEASRGPYATRQVDVSSLLVSGFGGGT |
| 1484 | Q92Y, S95A, | IYYPTTTSEGTFGGVAIAPGYTADASSLAWLAARLASHGF |
| | E136R, N154H, | VVFNIDTLTRFDYPDARGRQLLAALDYLTERSSVRGRVD |
| | N202C, F207I, | ASRLGVMGHSMGGGGTLRAVDGRPSLRAAVPLTPWHLD |
| | N210M, T247C | KTWPGVQTPTLIIGADNDTVAPVSSHAVPFYTSLPSSLDK |
| | | AYLELCNATHIAPMTTNTTIGKYAVAWMKRFVDDDTRY |
| | | DRFLCPGPSRGLCVEEYRSTCPF |
| | | (SEQ ID NO: 26) |
| SIBER | K64A, L90F, | AAVHGPDPTDALLEASRGPYATRQVDVSSLLVSGFGGGT |
| 1491 | Q92Y, E136R, | IYYPTTTSEGTFGGVAIAPGYTADASSLAWLAARLASHGF |
| | N154H, | VVFNIDTLTRFDYPDSRGRQLLAALDYLTERSSVRGRVD |
| | N202C, F207I, | ASRLGVMGHSMGGGGTLRAVDGRPSLRAAVPLTPWHLD |
| | N210M, T212A, | KTWPGVQTPTLIIGADNDTVAPVSSHAVPFYTSLPSSLDK |
| | T247C | AYLELCNATHIAPMTANTTIGKYAVAWMKRFVDDDTRY |
| | | DRFLCPGPSRGLCVEEYRSTCPF |
| | | (SEQ ID NO: 27) |
| SIBER | K64A, L90F, | AAVHGPDPTDALLEASRGPYATRQVDVSSLLVSGFGGGT |
| 1492 | Q92Y, E136R, | IYYPTTTSEGTFGGVAIAPGYTADASSLAWLAARLASHGF |
| | N154H, | VVFNIDTLTRFDYPDSRGRQLLAALDYLTERSSVRGRVD |
| | N202C, F207I, | ASRLGVMGHSMGGGGTLRAVDGRPSLRAAVPLTPWHLD |
| | N210M, G217A, | KTWPGVQTPTLIIGADNDTVAPVSSHAVPFYTSLPSSLDK |
| | T247C | AYLELCNATHIAPMTTNTTIAKYAVAWMKRFVDDDTRY |
| | | DRFLCPGPSRGLCVEEYRSTCPF |
| | | (SEQ ID NO: 28) |
| SIBER | K64A, L90F, | AAVHGPDPTDALLEASRGPYATRQVDVSSLLVSGFGGGT |
| 1493 | Q92Y, E136R, | IYYPTTTSEGTFGGVAIAPGYTADASSLAWLAARLASHGF |
| | N154H, | VVFNIDTLTRFDYPDSRGRQLLAALDYLTERSSVRGRVD |
| | N202C, F207I, | ASRLGVMGHSMGGGGTLRAVDGRPSLRAAVPLTPWHLD |
| | N210M, A220V, | KTWPGVQTPTLIIGADNDTVAPVSSHAVPFYTSLPSSLDK |
| | T247C | AYLELCNATHIAPMTTNTTIGKYVVAWMKRFVDDDTRY |
| | | DRFLCPGPSRGLCVEEYRSTCPF |
| | | (SEQ ID NO: 29) |
| SIBER | K64A, L90F, | AAVHGPDPTDALLEASRGPYATRQVDVSSLLVSGFGGGT |
| 1495 | Q92Y, E136R, | IYYPTTTSEGTFGGVAIAPGYTADASSLAWLAARLASHGF |
| | N154H, | VVFNIDTLTRFDYPDSRGRQLLAALDYLTERSSVRGRVD |
| | N202C, F207I, | ASRLGVMGHSMGGGGTLRAVDGRPSLRAAVPLTPWHLD |
| | N210M, L246A, | KTWPGVQTPTLIIGADNDTVAPVSSHAVPFYTSLPSSLDK |
| | T247C | AYLELCNATHIAPMTTNTTIGKYAVAWMKRFVDDDTRY |
| | | DRFLCPGPSRGACVEEYRSTCPF |
| | | (SEQ ID NO: 30) |

TABLE 4
Exemplary fusion tags

| | |
|---|---|
| His | MGSSHHHHHHAGA (SEQ ID NO: 31) |
| SUMO | MGGGSSEHEHEHSSGLVPRGSHMASMSDSEVNQEAKPEVKPE |
| | VKPETHINLKVSDGSSEIFFKIKKTTPLRRLMEAFAKRQGKEMD |
| | SLRFLYDGIRIQADQTPEDLDMEDNDIIEAHREQIGGSAGAETAS |
| | SEGLAGA (SEQ ID NO: 32) |
| NT11 | MVSEPHDYNYEKAGA (SEQ ID NO: 33) |
| MBP | MQLSHEHEHEHEHEHEHEGASGMKIEEGKVVIWHAMQPNELE |
| | VFQSLAEEYMALCPEVEIVFEQKPNLEDALKAAIPTGQGPDLFI |
| | WAHDWIGKFAEAGLLEPIDEYVTEDLLNEFAPMAQDAMQYKG |
| | HYYALPFAAETVAIIYNKEMVSEPPKTFDEMKAIMEKYYDPAN |
| | EKYGIAWPINAYFISAIAQAFGGYYFDDKTEQPGLDKPETIEGF |
| | KFFFTEIWPYMAPTGDYNTQQSIFLEGRAPMMVNGPWSINDVK |
| | KAGINFGVVPLPPIIKDGKEYWPRPYGGVKLIYFAAGIKNKDAA |
| | WKFAKWLTTSEESIKTLALELGYIPVLTKVLDDPEIKNDPVIYG |
| | FGQAVQHAYLMPKSPKMSAVWGGVDGAINEILQDPQNADIEGI |
| | LKKYQQEILNNMQGEENLYFQSIAGA |
| | (SEQ ID NO: 34) |
| PolyEK | AGAEKEKEKEKEKEKEKEKEKEKEKEKEKEKEKEKEKEKEKE |
| | KEKEKEKEKEKEKEKEKEKEKEKEKEKEKEKEKEKEKEKEKE |
| | KEKEKEKEK |
| | (SEQ ID NO: 35) |
| HA | MYPYDVPDYAAGA (SEQ ID NO: 36) |
| VNp15 | DVFKKGFSIADEGVVGAVE (SEQ ID NO: 37) |

It will be appreciated that many further modifications and permutations of various aspects of the described embodiments are possible. Accordingly, the described aspects are intended to embrace all such alterations, modifications, and variations that fall within the spirit and scope of the appended claims.

1. An isolated polypeptide that is distinguished from a wild-type hydrolase enzyme by at least one amino acid substitution, wherein the at least one amino acid substitution is at a position corresponding to position 202, 247, 207, 210, 64, 90, 92, 154, 61 or 212 of SEQ ID NO: 1.

2. The polypeptide of claim 1, wherein the polypeptide further comprises one or more amino acid substitutions at a position corresponding to position 4, 30, 31, 95, 136, 217, 220 or 246 of SEQ ID NO: 1.

3. The polypeptide of claim 1, wherein the polypeptide comprises an amino acid substitution at positions corresponding to one of the following sets of positions of SEQ ID NO: 1:
(a) 202 and 247;
(b) 202, 207 and 247;
(c) 202, 210 and 247;
(d) 202, 207, 210 and 247;
(e) 4, 202, 207, 210 and 247;
(f) 30, 202, 207, 210 and 247;
(g) 31, 202, 207, 210 and 247;
(h) 61, 202, 207, 210 and 247;
(i) 64, 202, 207, 210 and 247;
(j) 90, 202, 207, 210 and 247;
(k) 92, 202, 207, 210 and 247;
(l) 95, 202, 207, 210 and 247;
(m) 136, 202, 207, 210 and 247;
(n) 154, 202, 207, 210 and 247;
(o) 202, 207, 210, 212 and 247;
(p) 202, 207, 210, 217 and 247;
(q) 202, 207, 210, 220 and 247;
(r) 202, 207, 210, 246 and 247;
(s) 64, 90, 92, 136 and 154;
(t) 4, 64, 90, 92, 136 and 154;
(u) 30, 64, 90, 92, 136 and 154;
(v) 31, 64, 90, 92, 136 and 154;
(w) 61, 64, 90, 92, 136 and 154;
(x) 64, 90, 92, 95, 136 and 154;
(y) 64, 90, 92, 136, 154 and 212;
(z) 64, 90, 92, 136, 154 and 217;
(aa) 64, 90, 92, 136, 154 and 220;
(bb) 64, 90, 92, 136, 154 and 246;
(cc) 64, 90, 92, 136, 154, 202 and 247;
(dd) 64, 90, 92, 136, 154, 202, 207 and 247;
(ee) 64, 90, 92, 136, 154, 202, 210 and 247;
(ff) 64, 90, 92, 136, 154, 202, 207, 210 and 247;
(gg) 4, 30, 31, 61, 95, 212, 217, 220 and 246;
(hh) 4, 30, 31, 61, 64, 95, 212, 217, 220 and 246;
(ii) 4, 30, 31, 61, 90, 95, 212, 217, 220 and 246;
(jj) 4, 30, 31, 61, 92, 95, 212, 217, 220 and 246;
(kk) 4, 30, 31, 61, 95, 136, 212, 217, 220 and 246;
(ll) 4, 30, 31, 61, 95, 154, 212, 217, 220 and 246;
(mm) 4, 30, 31, 61, 95, 202, 212, 217, 220, 246 and 247;
(nn) 4, 30, 31, 61, 95, 207, 212, 217, 220 and 246;
(oo) 4, 30, 31, 61, 95, 210, 212, 217, 220 and 246;
(pp) 4, 64, 90, 92, 136, 154, 202, 207, 210 and 247;
(qq) 30, 64, 90, 92, 136, 154, 202, 207, 210 and 247;
(rr) 31, 64, 90, 92, 136, 154, 202, 207, 210 and 247;
(ss) 61, 64, 90, 92, 136, 154, 202, 207, 210 and 247;
(tt) 64, 90, 92, 95, 136, 154, 202, 207, 210 and 247;
(uu) 64, 90, 92, 136, 154, 202, 207, 210, 212 and 247;
(vv) 64, 90, 92, 136, 154, 202, 207, 210, 217 and 247;
(ww) 64, 90, 92, 136, 154, 202, 207, 210, 220 and 247;
(xx) 64, 90, 92, 136, 154, 202, 207, 210, 246 and 247;
(yy) 4, 30, 31, 61, 95, 202, 207, 210, 212, 217, 220, 246 and 247;
(zz) 4, 30, 31, 61, 64, 90, 92, 95, 136, 154, 212, 217, 220 and 246; and
(aaa) 4, 30, 31, 61, 64, 90, 92, 95, 136, 154, 202, 207, 210, 212, 217, 220, 246 and 247.

4. The polypeptide of claim 1, wherein the polypeptide comprises one or more amino acid substitutions selected from the following:
(a) a substitution to K, L or I at a position corresponding to position 4 of SEQ ID NO: 1;
(b) a substitution to G, A, V or S at a position corresponding to position 30 of SEQ ID NO: 1;
(c) a substitution to G, A, V, S or T at a position corresponding to position 31 of SEQ ID NO: 1;
(d) a substitution to G, A, V, I or S at a position corresponding to position 61 of SEQ ID NO: 1;
(e) a substitution to G, A, V, I, L or S at a position corresponding to position 64 of SEQ ID NO: 1;
(f) a substitution to M, S, T, F, Y, W, D or E at a position corresponding to position 90 of SEQ ID NO: 1;
(g) a substitution to G, A, V, I, L, S, M, P, F, Y, W or N at a position corresponding to position 92 of SEQ ID NO: 1;
(h) a substitution to G, A, V, I or T at a position corresponding to position 95 of SEQ ID NO: 1;
(i) a substitution to G, A, V, S, H, R or K at a position corresponding to position 136 of SEQ ID NO: 1;
(j) a substitution to G, A, S, T or H at a position corresponding to position 154 of SEQ ID NO: 1;
(k) a substitution to C or A at a position corresponding to position 202 of SEQ ID NO: 1;
(l) a substitution to G, A, V, I, L, S, M, T, P, F, Y or W at a position corresponding to position 207 of SEQ ID NO: 1;
(m) a substitution to G, A, V, I, L, S or M at a position corresponding to position 210 of SEQ ID NO: 1;
(n) a substitution to G, A, V, I, L or S at a position corresponding to position 212 of SEQ ID NO: 1;
(o) a substitution to A, V, I, S or T at a position corresponding to position 217 of SEQ ID NO: 1;
(p) a substitution to V, I, L, S or T at a position corresponding to position 220 of SEQ ID NO: 1;
(q) a substitution to G, A, V or S at a position corresponding to position 246 of SEQ ID NO: 1; and
(r) a substitution to C or A at a position corresponding to position 247 of SEQ ID NO: 1.

5. The polypeptide of claim 4, wherein the polypeptide comprises one or more amino acid substitutions selected from the following:
(a) a K substitution at a position corresponding to position 4 of SEQ ID NO: 1;
(b) a A substitution at a position corresponding to position 30 of SEQ ID NO: 1;
(c) a A substitution at a position corresponding to position 31 of SEQ ID NO: 1;
(d) a G substitution at a position corresponding to position 61 of SEQ ID NO: 1;
(e) a A substitution at a position corresponding to position 64 of SEQ ID NO: 1;
(f) a F substitution at a position corresponding to position 90 of SEQ ID NO: 1;
(g) a Y or G substitution at a position corresponding to position 92 of SEQ ID NO: 1;
(h) a A substitution at a position corresponding to position 95 of SEQ ID NO: 1;
(i) a R substitution at a position corresponding to position 136 of SEQ ID NO: 1;
(j) a H substitution at a position corresponding to position 154 of SEQ ID NO: 1;
(k) a C substitution at positions corresponding to positions 202 and 247 of SEQ ID NO: 1;
(l) a I substitution at a position corresponding to position 207 of SEQ ID NO: 1;
(m) a M substitution at a position corresponding to position 210 of SEQ ID NO: 1;
(n) a A substitution at a position corresponding to position 212 of SEQ ID NO: 1;
(o) a A substitution at a position corresponding to position 217 of SEQ ID NO: 1;
(p) a V substitution at a position corresponding to position 220 of SEQ ID NO: 1; and
(q) a A substitution at a position corresponding to position 246 of SEQ ID NO: 1.

6. The polypeptide of claim 5, wherein the polypeptide comprises one or more of the following amino acid substitutions:
(a) a C substitution at positions corresponding to positions 202 and 247 of SEQ ID NO: 1;
(b) a I substitution at a position corresponding to position 207 of SEQ ID NO: 1; and
(c) a M substitution at a position corresponding to position 210 of SEQ ID NO: 1.

7. The polypeptide of claim 5, wherein the polypeptide comprises one or more of the following amino acid substitutions:
(a) a A substitution at a position corresponding to position 64 of SEQ ID NO: 1;
(b) a F substitution at a position corresponding to position 90 of SEQ ID NO: 1;
(c) a Y or G substitution at a position corresponding to position 92 of SEQ ID NO: 1;
(d) a R substitution at a position corresponding to position 136 of SEQ ID NO: 1; and
(e) a H substitution at a position corresponding to position 154 of SEQ ID NO: 1.

8. The polypeptide of claim 5, wherein the polypeptide comprises one or more of the following amino acid substitutions:
(a) a K substitution at a position corresponding to position 4 of SEQ ID NO: 1;
(b) a A substitution at a position corresponding to position 30 of SEQ ID NO: 1;
(c) a A substitution at a position corresponding to position 31 of SEQ ID NO: 1;
(d) a G substitution at a position corresponding to position 61 of SEQ ID NO: 1;
(e) a A substitution at a position corresponding to position 95 of SEQ ID NO: 1;
(f) a A substitution at a position corresponding to position 212 of SEQ ID NO: 1;
(g) an A substitution at a position corresponding to position 217 of SEQ ID NO: 1;
(h) a V substitution at a position corresponding to position 220 of SEQ ID NO: 1; and
(i) an A substitution at a position corresponding to position 246 of SEQ ID NO: 1.

9. The polypeptide of claim 1, wherein the wild-type hydrolase enzyme comprises an amino acid sequence having at least 70% sequence identity to an amino acid sequence in SEQ ID NO: 1-7.

10. The polypeptide of claim 9, wherein the polypeptide comprises an amino acid sequence with at least 70% sequence identity to an amino acid sequence set forth in SEQ ID NO: 14-30.

11. The polypeptide of claim 1, wherein the polypeptide has hydrolase activity, optionally wherein the polypeptide is capable of degrading a polyester, further optionally wherein the polypeptide is capable of degrading PET.

12. (canceled)

13. (canceled)

14. The polypeptide of claim 1, wherein the polypeptide comprises a fusion partner, optionally wherein the fusion partner comprises an amino acid sequence set forth in any one of SEQ ID NO: 31-37.

15. (canceled)

16. An isolated polypeptide comprising an amino acid sequence having at least 70% sequence identity to an amino acid sequence set forth in SEQ ID NO: 1, SEQ ID NO: 2, SEQ ID NO: 3 or SEQ ID NO: 4, SEQ ID NO: 5, SEQ ID NO: 6 or SEQ ID NO: 7.

17. The polypeptide of claim 16, wherein the polypeptide comprises an amino acid sequence set forth in SEQ ID NO: 1, SEQ ID NO: 2, SEQ ID NO: 3 or SEQ ID NO: 4, SEQ ID NO: 5, SEQ ID NO: 6, or SEQ ID NO: 7.

18. The polypeptide of claim 16, wherein the polypeptide further comprises an amino acid substitution at one or more positions corresponding to position 4, 30, 31, 61, 64, 90, 92, 95, 136, 154, 202, 207, 210, 212, 217, 220, 246 or 247 of SEQ ID NO: 1.

19. An isolated polypeptide comprising an amino acid sequence that is distinguished from an amino acid sequence of SEQ ID NO: 1-7 by at least one amino acid substitution, wherein the at least one amino acid substitution is at a position corresponding to position 4, 30, 31, 61, 64, 90, 92, 95, 136, 154, 202, 207, 210, 212, 217, 220, 246 or 247 of SEQ ID NO: 1.

20-31. (canceled)
